(19) United States
(12) Patent Application Publication
(10) Pub. No.: US 2018/0289780 A1
(43) Pub. Date: Oct. 11, 2018

(54) OSTEOPONTIN AND THROMBIN-CLEAVED FRAGMENT THEREOF AND THEIR USE IN ATHEROSCLEROSIS PREVENTION, INFLAMMATION REDUCTION AND IMPROVING PLAQUE STABILITY

(71) Applicant: CardioVax, LLC, Los Angeles, CA (US)

(72) Inventors: Jan Nilsson, Genarp (SE); Claes Pontus Duner, Dalby (SE)

(73) Assignee: CardioVax, LLC, Los Angeles, CA (US)

(21) Appl. No.: 15/947,591

(22) Filed: Apr. 6, 2018

Related U.S. Application Data
(60) Provisional application No. 62/482,787, filed on Apr. 7, 2017.

(51) Int. Cl. A61K 39/00; A61K 47/64; A61K 9/00; A61P 9/10; A61K 39/39

(52) U.S. Cl. CPC A61K 39/0005; A61K 47/643; A61K 9/0019; A61K 47/42; A61K 9/0053; A61K 9/0043; A61K 39/39; A61P 9/10

(57) ABSTRACT

Described herein are compositions comprising osteopontin (OPN) or an antigenic fragment thereof and a protein carrier and uses of the compositions to treat cardiovascular diseases.

### CROSS-REFERENCE TO RELATED APPLICATIONS

This application includes a claim of priority under 35 U.S.C. § 119(e) to U.S. provisional patent application No. 62/482,787, filed Apr. 7, 2017, the entirety of which is hereby incorporated by reference.

### REFERENCE TO SEQUENCE LISTING

The Sequence Listing submitted Apr. 6, 2018, as a text file named “SequenceListing-070017-000026US00_ST25” created on Apr. 6, 2018 and having a size of 2,275 bytes, is hereby incorporated by reference.

### FIELD OF INVENTION

Provided herein are compositions and method for treating or preventing cardiovascular diseases.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Osteopontin (OPN) is a multifunctional protein that is highly expressed in chronic inflammatory diseases, including rheumatoid arthritis, systemic lupus erythematosus, psoriasis and atherosclerosis. Clinical studies have demonstrated increased OPN levels in plasma of patients with cardiovascular disease and OPN has been proposed as a predictor of coronary artery disease, restenosis and aortic aneurysms. Experimental studies using OPN^{-/-} mice suggest that OPN is an enhancer of atherosclerotic plaque formation by increasing plaque inflammation.

Structurally, the OPN molecule contains several cell-interacting domains, as well as a thrombin cleavage site, which have been demonstrated to regulate its activity. These domains include the integrin-binding RGD sequence, a CD44 interaction site, and a cryptic SLAYGLR (SEQ ID NO: 1) (SVVYGLR, SEQ ID NO:2, in human) site that is exposed after cleavage of OPN by thrombin (FIG. 1A). The SLAYGLR (SEQ ID NO: 1) domain interacts with integrin α9β1, α4β1, and α4β7 (FIG. 1A) and mediates cell adhesion to the N-terminal fragment of thrombin cleaved OPN (tcOPN), causing cell signaling in an RGD-independent manner. The α9β1, α4β1, and α4β7 integrins are expressed on multiple immune cells including monocytes, macrophages and neutrophils, and ligation of these integrins affects important cellular functions like adhesion and migration. Previous studies have demonstrated that increased levels of the N-terminal fragment of tcOPN in carotid plaques are associated with enhanced plaque inflammation in hypertensive patients and that statin treatment is associated with lower plaque content of tcOPN and increased plaque stability. Furthermore, blocking antibodies against the cryptic SLAYGLR (SEQ ID NO: 1) site of tcOPN inhibited in vitro monocyte migration as well as in vivo inflammatory cell infiltration in joints of arthritic mice.

It is an objective of the present invention to provide a composition that induces blocking antibodies against the integrin-interacting domain(s) of OPN and mitigates plaque inflammation.

It is another objective of the present invention to provide a method of treating or reducing the likelihood or severity of atherosclerosis, inflammation and cardiovascular diseases.

### SUMMARY

The following embodiments and aspects thereof are described and illustrated in conjunction with systems, compositions and methods which are meant to be exemplary and illustrative, not limiting in scope.

Provided herein are compositions including osteopontin (OPN) or an antigenic fragment thereof and a protein carrier. In some embodiments, the OPN or the antigenic fragment thereof is fused to a protein carrier.

Exemplary protein carriers include bovine serum albumin (BSA), nonalbumin, edestin, exoprotein A from Pseudomonas aeruginosa, HC (hemocyanin from crab Paralithodes camtschatica), Helix promatia haemocyanin (HPH), human serum albumin (HSA), KTI (Kunits trypsin inhibitor from soybeans), keyhole limpet haemocyanin (KLH), LPH (haemocyanin from Limulus polyphemus), ovalbumin, Pam3Cys-Th, polylysine, porcine thyroglobulin (PTG), purified protein derivative (PPD), rabbit serum albumin (RSA), soybean trypsin inhibitor (STI), sunflower globulin (SFG), tetanus toxoid, diphtheria toxoid, PADRE (pan HLA DR-bindin epitope), Haemophilus influenza protein D, Neisseria meningitides outer membrane protein, flagellin, or CRM197. Preferably, the protein carrier contains an epitope that induces strong antibody responses.

In one embodiment, the fragment of OPN includes and not limited to, consists essentially of, or consists of the sequence SLAYGLR (SEQ ID NO:1) and the protein carrier is or includes PADRE, which has a sequence of AKFVAAWTLKAAA (SEQ ID NO:3).

In one embodiment of the composition, the antigen fragment consists of an amino acid sequence at least 85% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In another embodiment, the antigen fragment consists of an amino acid sequence at least 90% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In a further embodiment, the antigen fragment consists of an amino acid sequence at least 95% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In some embodiments, the fragment of OPN induces an antibody response.

In other embodiments, a composition is provided including a human OPN, or an antigenic fragment thereof being (i.e., consisting of or consisting essentially of) (i.e., including but not limited to) the sequence SVVYGLR (SEQ ID NO:2) or an epitope containing SVVYGLR (SEQ ID NO:2), such as GRGDSVVYGLR (SEQ ID NO: 8) or VDTYDGRGDSVVYGLR (SEQ ID NO: 9), and a protein carrier. In some aspects, the human OPN or an antigenic fragment thereof is fused with a carrier protein or peptide, such as PADRE, which induces strong antibody responses in vivo. In other aspects, the composition includes human OPN or an antigenic fragment thereof, a protein carrier (preferably inducing strong antibody responses), and an adjuvant. An exemplary adjuvant is alum, i.e., a hydrated double sulfate salt of aluminum.

Provided herein are methods for treating, inhibiting or reducing the severity of cardiovascular diseases in a subject in need thereof. The methods include administering to the subject, therapeutically effective amounts of compositions comprising osteopontin (OPN) or an antigenic fragment thereof and a protein carrier. In one embodiment, the composition in the methods comprises a peptide containing the sequence SLAYGLR (SEQ ID NO:1) and PADRE as the protein carrier. In another embodiment, the composition in the methods comprises a peptide containing the sequence SVVYGLR (SEQ ID NO:2), such as GRGDSVVYGLR (SEQ ID NO: 8) or VDTYDGRGDSVVYGLR (SEQ ID NO: 9), and PADRE as the protein carrier. In yet another embodiment, the composition in the methods comprises a peptide containing the sequence SLAYGLR (SEQ ID NO:1) or SVVYGLR (SEQ ID NO:2), which is fused with PADRE as the protein carrier. In some embodiments, the administration is performed via an oral, nasal, subcutaneous, or intramuscular route of administration. In some embodiments, the subject has or shows symptoms of diabetes.

Provided herein are methods for preventing or reducing the likelihood of cardiovascular diseases in a subject in need thereof. The methods include administering to the subject, therapeutically effective amounts of compositions comprising osteopontin (OPN) or an antigenic fragment thereof and a protein carrier, as described herein. In one embodiment, the composition comprises a peptide having the sequence SLAYGLR (SEQ ID NO:1) and PADRE as the protein carrier. In some embodiments, the administration is performed via an oral, nasal, subcutaneous, or intramuscular route of administration.

Further provided herein is an immunogenic composition comprising the compositions described herein together with an adjuvant and/or an excipient.

A method is also provided of treating, reducing the likelihood or severity of, or slowing the progression of cardiovascular death in a subject, including detecting an elevated level of thrombin-cleaved osteopontin (tcOPN) in a carotid plaque of the subject; and administering a therapeutically effective amount of a composition containing osteopontin (OPN) or an antigenic fragment thereof and a protein carrier to the subject. In various embodiments, the subject has diabetes, or type 2 diabetes. Generally, the elevated level of tcOPN is higher than an averaged value obtained from subjects without diabetes or from asymptomatic subjects.

### BRIEF DESCRIPTION OF THE DRAWINGS

Exemplary embodiments are illustrated in referenced figures. It is intended that the embodiments and figures disclosed herein are to be considered illustrative rather than restrictive.

FIGS. 1A-1D depicts in accordance with various embodiments of the invention, that high fat diet (HFD) increases tcOPN levels in plasma. FIG. 1A is an schematic illustration of the OPN protein indicating thrombin cleavage site, RGD sequence, the cryptic SLAYGLR sequence that is exposed after cleavage by thrombin, and integrins α9β1, α4β1, and α4β7 interacting with the SLAYGLR (SEQ ID NO: 1) domain. FIG. 1B is an schematic illustration of the experimental design during the study on early and advanced atherosclerosis. FIG. 1C depicts HFD increased OPN, and FIG. 1D depicts thrombin cleaved OPN levels in plasma.

FIGS. 2A-2H depict immunization of ApoE^{-/-} mice with fused tcOPN-PADRE results in IgG against tcOPN and tcOPN-PADRE and decreased tcOPN in plasma. In FIGS. 2A-2D, ApoE^{-/-} mice were immunized with tcOPN-PADRE or PADRE and kept on HFD for 8 weeks before sacrifice. Plasma (dilution 1:100 or 1:1000) from immunized mice were analyzed for IgG2a (2A for PADRE immunized mice; 2B for tcOPN-PADRE immunized mice) or IgG1 (2C for PADRE immunized mice; 2D for tcOPN-PADRE immunized mice) against PADRE peptide, tcOPN-PADRE peptide, OPN protein and tcOPN protein. FIGS. 2E and 2F show the amounts of IgG2a (2E) and IgG1 (2F) against tcOPN-PADRE peptide in plasma (dilution 1:1000) from mice immunized with PADRE or tcOPN-PADRE and kept on HFD for 15 weeks. Figures G and H show the levels of OPN (2G) and tcOPN (2H) protein in plasma in mice immunized with tcOPN-PADRE peptide or PADRE and kept on HFD for 8 weeks (early) and 15 weeks (advanced). Data are shown as (A-D): mean±SD, n=3 (pooled plasma for the different groups), (E-F): mean±SD, n=13-14 (plasma from individual mice) and G: mean±SD, n=8 for 8 weeks of HFD and n=13-14 for 15 weeks of HFD (plasma from individual mice).

FIGS. 3A-3C depict immunization of ApoE^{-/-} mice with tcOPN reduces inflammatory monocytes in spleen. Splenocytes from mice immunized with tcOPN-PADRE peptide or PADRE peptide and kept on HFD for 8 weeks were analyzed for inflammatory Ly6c^{Hi} monocytes (3A) and expression of α4 (3B) and α9 (3C) integrin's on the Ly6c^{Hi} monocyte population.

FIGS. 4A-4F depict immunization of ApoE^{-/-} mice with tcOPN reduces early and advanced atherosclerotic plaque development. En-face preparations of the descending aorta (4A, 4B), aortic arch (4C, 4D) and subvalvular plaques (4E, 4F) were analyzed for plaque content or plaque area in mice immunized with tcOPN-PADRE peptide or PADRE peptide and kept on HFD for 8 (4A, 4C, 4E) or 15 weeks (4B, 4D, 4F).

FIGS. 5A-5C depict immunization of ApoE^{-/-} mice with tcOPN-PADRE increases plaque stability and reduces inflammatory monocytes in subvalvular plaques. Subvalvular plaques from mice immunized with tcOPN-PADRE peptide or PADRE peptide and kept on HFD for 8 weeks were analyzed for (5A) smooth muscle cell α-actin, (5B) collagen (van Gieson) and (5C) inflammatory monocytes (Ly6C) positive plaque area. Data are presented as mean±SD percentage positive staining of plaque area, n=8. Representative sections are included for each staining. Scale bar=350 μm. *P<0.05, *P<0.01.

FIGS. 6A-6F depict monocyte migration towards tcOPN is inhibited by plasma from tcOPN immunized mice. FIGS. 6A-6C show inflammatory Ly-6c^{hi} monocytes, and their expression of integrin α4 and α9, are increased in ApoE^{-/-} mice on HFD. FIG. 6D shows blood monocytes from ApoE^{-/-} mice migrate towards tcOPN, but not towards full-length OPN in vitro. FIG. 6E shows plasma from mice immunized with tcOPN-PADRE blocks the migratory capacity of monocytes towards tcOPN. FIG. 6F shows α9β1, α4β1 and α4β7 integrin binding to tcOPN peptide is inhibited with plasma from mice immunized with tcOPN-PADRE.

FIGS. 7A-7F depict tcOPN-PADRE immunization has limited influence on the T cell populations. Flow cytometry analysis of Th1 (7A, 7B), Th2 (7C, 7D) and Treg (7E, 7F) splenocytes in ApoE^{-/-} mice immunized with tcOPN-PADRE or PADRE in early (7A, 7C, 7E) and late (7B, 7D, 7F) atherosclerosis.

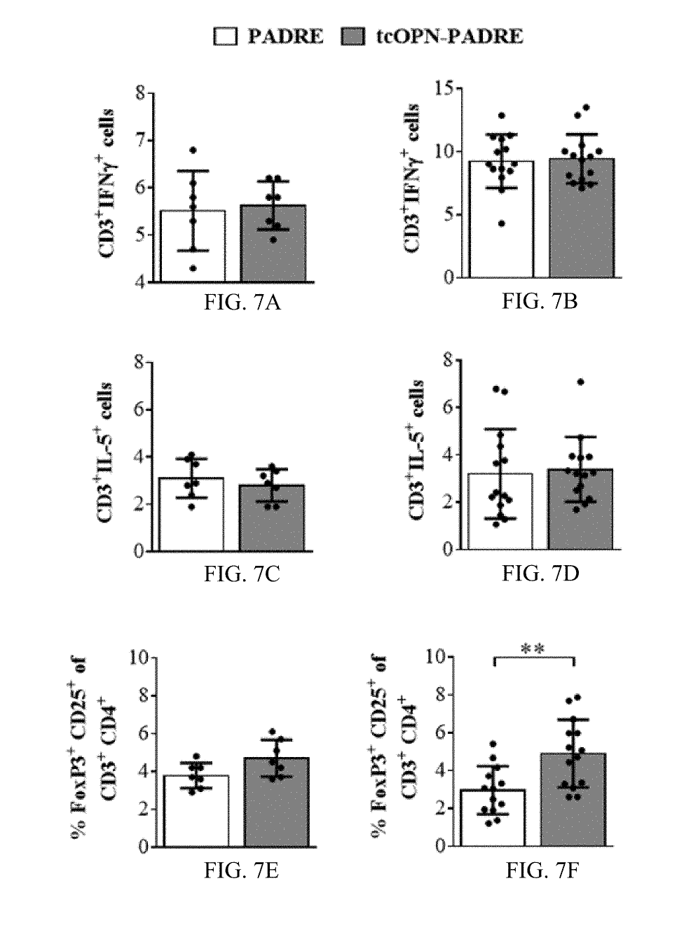

FIGS. 8A and 8B depict immunization of ApoE^{-/-} mice with tcOPN reduces atherosclerotic plaque development. En-face preparations of the aortic arch (8A) and subvalvular plaques (8B) were analyzed for percentage plaque content or plaque area in mice immunized with tcOPN-PADRE peptide or PADRE peptide.

FIG. 9 depicts in accordance with various embodiments of the invention, THP-1 monocytes migrate against tcOPN. THP-1 monocytes display increased migration towards tcOPN in vitro.

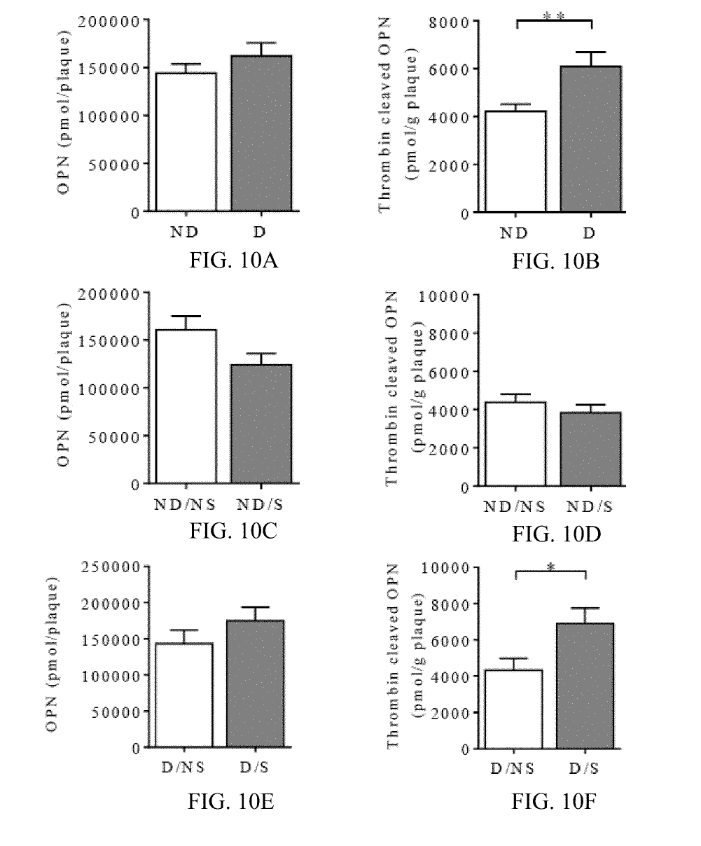

FIGS. 10A-10F depict the levels of OPN and tcOPN in human coronary plaques from the study in Example 2. The level of OPN in plaques from non-diabetic (ND) and diabetic (D) patients did not differ (10A). Plaques from diabetic patients contained significantly more thrombin cleaved OPN compared to ND (10B). There was no difference in either OPN (10C) or thrombin cleaved OPN (10D) levels in ND subjects when dividing plaques into non-symptomatic (NS) and symptomatic plaques (S). In the diabetic cohort, thrombin cleaved OPN (10F), but not OPN (10E), was significantly increased in plaques from symptomatic patients compared no non-symptomatic.

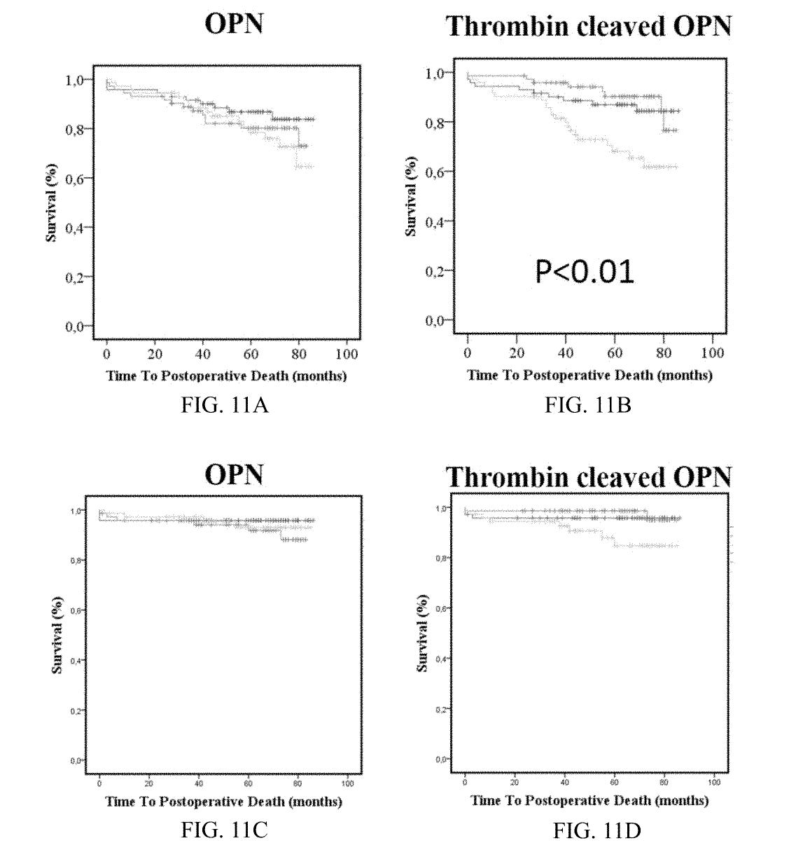

FIGS. 11A-11F depict Kaplan-Meier plots of event-free survival in the study in Example 2 with population divided into tertiles according to OPN or tcOPN basal levels at time of operation and total death (11A and 11B, respectively), cardiovascular death (11C and 11D, respectively) and cardiovascular event risk (11E and 11F, respectively). A significant positive linear trend over tertiles was detected using a log-rank test.

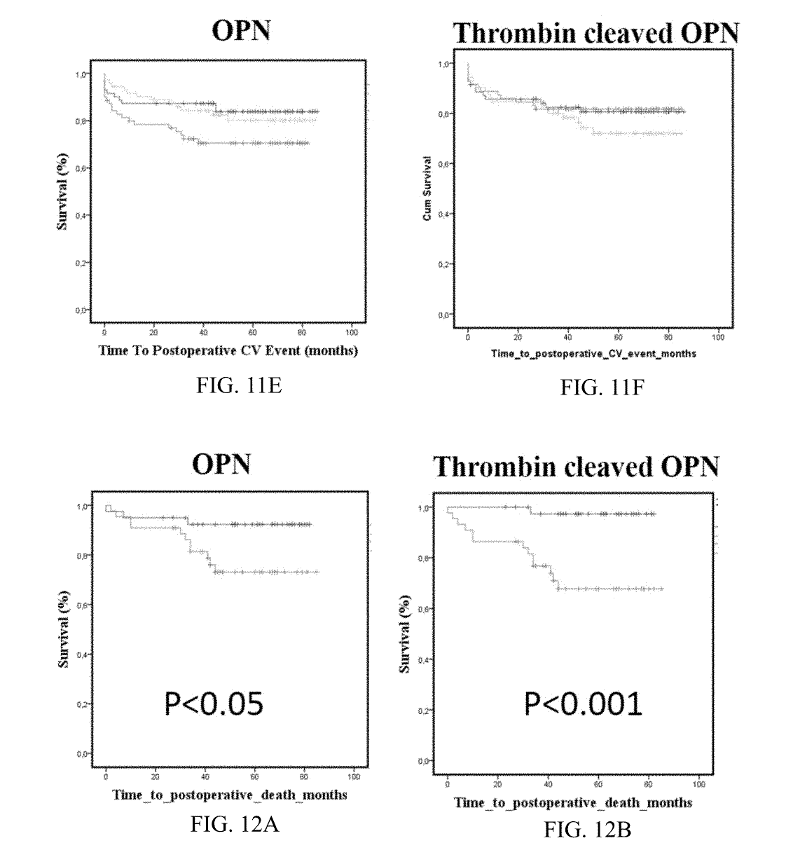

FIGS. 12A-12F depict Kaplan-Meier plots of event-free survival in the diabetic study population divided into two-tiles according to OPN or tcOPN basal levels at time of operation and total death (12A and 12B, respectively), cardiovascular death (12C and 12D, respectively) and cardiovascular event risk (12E and 12F, respectively). A significant positive linear trend was detected using a log-rank test.

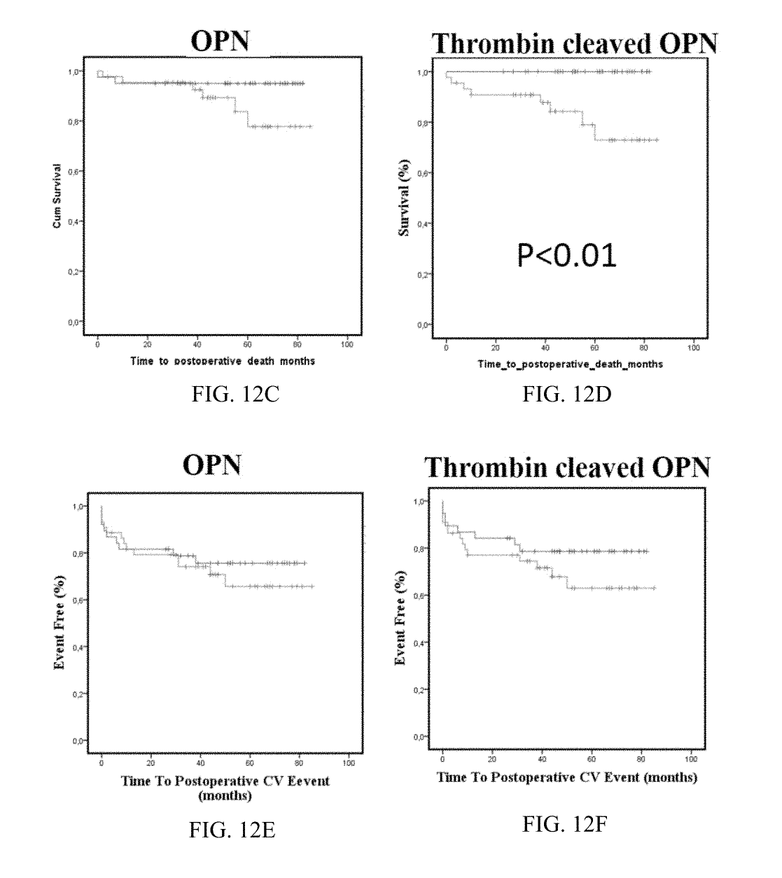

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, NY 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein the term “comprising” or “comprises” is used in reference to compositions, methods, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.).

“Administering” and/or “administer” as used herein refer to any route for delivering a pharmaceutical composition to a patient. Routes of delivery may include non-invasive peroral (through the mouth), topical (skin), transmucosal (nasal, buccal/sublingual, vaginal, ocular and rectal) and inhalation routes, as well as parenteral routes, and other methods known in the art. Parenteral refers to a route of delivery that is generally associated with injection, including intraorbital, infusion, intraarterial, intracarotid, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders.

“Beneficial results” may include, but are in no way limited to, lessening or alleviating the severity of the disease condition, preventing the disease condition from worsening, curing the disease condition, preventing the disease condition from developing, lowering the chances of a patient developing the disease condition and/or prolonging a patient's life or life expectancy. In some embodiments, the disease condition is cardiovascular disease. In some embodiments, the disease condition is atherosclerosis.

The term “effective amount” as used herein refers to the amount of a pharmaceutical composition comprising one or more peptides as disclosed herein or a mutant, variant, analog or derivative thereof, to decrease at least one or more symptom of the disease or disorder, and relates to a sufficient amount of pharmacological composition to provide the desired effect. The phrase “therapeutically effective amount” as used herein means a sufficient amount of the composition to treat a disorder, at a reasonable benefit/risk ratio applicable to any medical treatment. In one embodiment, the pharmaceutical (therapeutic) composition comprises, consists of or consists essentially of any one or more peptides described herein. In another embodiment, the pharmaceutical composition comprises a vaccine comprising, consisting of or consisting essentially of any one or more peptides described herein. In various embodiments, the pharmaceutical compositions described herein further comprise an adjuvant. In various embodiments, the pharmaceutical compositions described herein further comprise a pharmaceutically acceptable carrier. In some embodiments, a therapeutic pharmaceutical composition is used, for example, to treat, inhibit, reduce the severity of and/or, reduce duration of atherosclerosis and/or related symptoms in a subject in need thereof. In some embodiments, the vaccine is used to prevent atherosclerosis and/or related symptoms in a subject.

A therapeutically or prophylactically significant reduction in a symptom is, e.g. at least about 10%, at least about 20%, at least about 30%, at least about 40%, at least about 50%, at least about 60%, at least about 70%, at least about 80%, at least about 90%, at least about 100%, or more in a measured parameter as compared to a control or non-treated subject or the state of the subject prior to administering the compositions and/or vaccines described herein. Measured or measurable parameters include clinically detectable markers of disease, for example, elevated or depressed levels of a biological marker, as well as parameters related to a clinically accepted scale of symptoms or markers for cardiovascular diseases or atherosclerosis. It will be understood, however, that the total daily usage of the compositions and formulations as disclosed herein will be decided by the attending physician within the scope of sound medical judgment. The exact amount required will vary depending on factors such as the type of disease being treated, gender, age, and weight of the subject.

“Subject” or “individual” or “animal” or “patient” or “mammal,” is meant any subject, particularly a mammalian subject, for whom diagnosis, prognosis, or therapy is desired. Mammalian subjects include, but are not limited to, humans, domestic animals, farm animals, zoo animals, sport animals, pet animals such as dogs, cats, guinea pigs, rabbits, rats, mice, horses, cattle, cows; primates such as apes, monkeys, orangutans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. In certain embodiments, the mammal is a human subject.

As used herein, the terms “treat,” “treatment,” “treating,” or “amelioration” when used in reference to a disease, disorder or medical condition, refer to both therapeutic treatment and prophylactic or preventative measures, wherein the object is to prevent, reverse, alleviate, ameliorate, inhibit, lessen, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a disease-state is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the condition even if the treatment is ultimately unsuccessful. Those in need of treatment include those already with the condition as well as those prone to have the condition or those in whom the condition is to be prevented.

“Pharmaceutically acceptable carriers” as used herein refer to conventional pharmaceutically acceptable carriers useful in this invention.

“Peptidomimetic” as used herein is a small protein-like chain designed to mimic a protein function. They may be modifications of an existing peptide or newly designed to mimic known peptides. They may be, for example peptoids and/or β-peptides and/or D-peptides

The term “fusion protein” as used herein indicates a protein created through the attaching of two or more polypeptides which originated from separate proteins. In particular fusion proteins can be created by recombinant DNA technology and are typically used in biological research or therapeutics. Fusion proteins can also be created through chemical covalent conjugation with or without a linker between the polypeptides portion of the fusion proteins.

The term “attach” or “attached” as used herein, refers to connecting or uniting by a bond, link, force or tie in order to keep two or more components together, which encompasses either direct or indirect attachment such that for example where a first polypeptide is directly bound to a second polypeptide or material, and the embodiments wherein one or more intermediate compounds, and in particular polypeptides, are disposed between the first polypeptide and the second polypeptide or material.

The term “protein” or “polypeptide” as used herein indicates an organic polymer composed of two or more amino acid monomers and/or analogs thereof. The term “polypeptide” includes amino acid polymers of any length including full length proteins and peptides, as well as analogs and fragments thereof. A polypeptide of three or more amino acids is also called an oligopeptide. As used herein the terms “amino acid”, “amino acidic monomer”, or “amino acid residue” refer to any of the twenty naturally occurring amino acids including synthetic amino acids with unnatural side chains and including both D and L optical isomers. The term “amino acid analog” refers to an amino acid in which one or more individual atoms have been replaced, either with a different atom, isotope, or with a different functional group but is otherwise identical to its natural amino acid analog.

A peptide as disclosed herein includes conservative variants of that peptide. A conservative variant refers to a peptide that has at least one amino acid substituted by another amino acid or an amino acid analog that has at least one property similar to that of the original amino acid from an exemplary reference peptide. Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative substitution can be assessed by a variety of factors, such as, e.g., the physical properties of the amino acid being substituted (Table 1) or how the original amino acid would tolerate a substitution (Table 2). The selections of which amino acid can be substituted for another amino acid in a peptide disclosed herein are known to a person of ordinary skill in the art. A conservative variant can function in substantially the same manner as the exemplary reference peptide, and can be substituted for the exemplary reference peptide in any aspect of the present specification.

TABLE 1
Amino Acid Properties

| Property | Amino Acids |
|---|---|
| Aliphatic | G, A, I, L, M, P, V |
| Aromatic | F, H, W, Y |
| C-beta branched | I, V, T |
| Hydrophobic | C, F, I, L, M, V, W |
| Small polar | D, N, P |
| Small non-polar | A, C, G, S, T |
| Large polar | E, H, K, Q, R, W, Y |
| Large non-polar | F, I, L, M, V |
| Charged | D, E, H, K, R |
| Uncharged | C, S, T |
| Negative | D, E |
| Positive | H, K, R |
| Acidic | D, E |
| Basic | K, R |
| Amide | N, Q |

TABLE 2
Amino Acid Substitutions

| Amino | Favored | Neutral | Disfavored |
|---|---|---|---|
| Acid | Substitution | Substitutions | substitution |
| A | G, S, T | C, E, I, K, M, L, P, | D, F, H, N, Y, W |
| | | Q, R, V | |
| C | F, S, Y, W | A, H, I, M, L, T, V | D, E, G, K, N, P, Q, R |
| D | E, N | G, H, K, P, Q, R, S, | A, C, I, L, |
| | | T | |
| E | D, K, Q | A, H, N, P, R, S, T | C, F, G, I, L, M, V, W, |
| | | | Y |
| F | M, L, W, Y | C, I, V | A, D, E, G, H, K, N, P, |
| | | | Q, R, S, T |
| G | A, S | D, K, N, P, Q, R | C, E, F, H, I, L, M, T, |
| | | | V, W, Y |
| H | N, Y | C, D, E, K, Q, R, S, | A, F, G, I, L, M, P, V |
| | | T, W | |
| I | V, L, M | A, C, T, F, Y | D, E, G, H, K, N, P, Q, |
| | | | R, S, W |
| K | Q, E, R | A, D, G, H, M, N, P, | C, F, I, L, V, W, Y |
| | | S, T | |
| L | F, I, M, V | A, C, W, Y | D, E, G, H, K, N, P, Q, |
| | | | R, S, T |
| M | F, I, L, V | A, C, R, Q, K, T, W, | D, E, G, H, N, P, S |
| | | Y | |
| N | D, H, S | E, G, K, Q, R, T | A, C, F, I, L, M, P, V, |
| | | | W, Y |
| P | — | A, D, E, G, K, Q, R, | C, F, H, I, L, M, N, V, |
| | | S, T | W, Y |
| Q | E, K, R | A, D, G, H, M, N, P, | C, F, I, L, V, W, Y |
| | | S, T | |
| R | K, Q | A, D, E, G, H, M, N, | C, F, I, L, V, W, Y |
| | | P, S, T | |
| S | A, N, T | C, D, E, G, H, K, P, | F, I, L, M, V, W, Y |
| | | Q, R, T | |
| T | S | A, C, D, E, H, I, K, | F, G, L, W, Y |
| | | M, N, P, Q, R, V | |
| V | I, L, M | A, C, F, T, Y | D, E, G, H, K, N, P, Q, |
| | | | R, S, W |
| W | F, Y | H, L, M | A, C, D, E, G, I, K, N, |
| | | | P, Q, R, S, T, V |
| Y | F, H, W | C, I, L, M, V | A, D, E, G, K, N, P, Q, |
| | | | R, S, T |

Matthew J. Betts and Robert, B. Russell, Amino Acid Properties and Consequences of Substitutions, pp. 289-316, In Bioinformatics for Geneticists, (eds Michael R. Barnes, Ian C. Gray, Wiley, 2003).

The term “antigen”, as it is used herein, relates to any substance that, when introduced into the body can stimulate an immune response. Antigens comprise exogenous antigens (antigens that have entered the body from the outside, for example by inhalation, ingestion, or injection) and endogenous antigens or autoantigens (antigens that have been generated within the body). In particular, an “autoantigen” is an antigen that despite being a normal tissue constituent is the target of a humoral or cell-mediated immune response.

The term “regulatory T cell,” “T regulatory cell” or “Treg” as used herein indicates a component of the immune system that suppresses immune responses of other cells, and comprises T cells that express the CD8 transmembrane glycoprotein (CD8+ T cells); T cells that express CD4, CD25, and Foxp3 (CD4+CD25+ regulatory T cells); and other T cell types that have suppressive function identifiable by a skilled person. Treg comprise both naturally occurring T cells and T cells generated in vitro.

The term “antigen-specific” as used herein indicates an immunity response, and in particular, immunological tolerance, for a certain antigen which is characterized by a substantially less or no immune response (and in particular, immunological tolerance) for another antigen. Accordingly, an antigen-specific regulatory T cell, specific for one or more autoantigens is able, under appropriate conditions to minimize to the specific immune response to the one or more autoantigens with substantially less or no minimizing effect on the immune response towards other antigens or autoantigens.

Fusion proteins comprising autoantigen associated with atherogenesis and/or atherosclerosis and related methods and systems are herein described that are capable of eliciting an autoantigen-specific Treg response and that in several embodiments can be used for treating and/or preventing atherosclerosis or a condition associated thereto in an individual.

The term “atherosclerosis” as used herein indicates an inflammatory condition, and in particular the condition in which an artery wall thickens as the result of a build-up of fatty materials such as cholesterol. In some cases, atherosclerosis is treated with statin therapy (1). In several cases, atherosclerosis is a syndrome affecting arterial blood vessels, a chronic inflammatory response in the walls of arteries, in large part due to the accumulation of macrophage white blood cells and promoted by low-density lipoproteins (LDL, plasma proteins that carry cholesterol and triglycerides) without adequate removal of fats and cholesterol from the macrophages by functional high-density lipoproteins (HDL), (see apoA-1 Milano). Lipid retention and modification in the arterial intima in some cases elicit a chronic inflammatory process with autoimmune responses and the development of atherosclerotic lesions (2). Both adaptive and innate immune mechanisms have been described as contributors to this process (3-6). While pattern recognition receptors of innate immunity are believed to account for cholesterol uptake and contribute to activation of macrophages and endothelial cells, antigen-specific T cells recognizing LDL particles in the intima provide strong pro-inflammatory stimuli that accelerate atherogenesis. Atherosclerosis is commonly referred to as a hardening or furring of the arteries. It is believed to be caused by the formation of multiple plaques within the arteries. Typically, autoimmune responses to LDL contribute to its progression, while immunization with LDL may induce atheroprotective or proatherogenic responses.

The term “condition” as used herein indicates as usually the physical status of the body of an individual (as a whole or of one or more of its parts) that does not conform to a physical status of the individual (as a whole or of one or more of its parts) that is associated with a state of complete physical, mental and possibly social well-being. Conditions herein described include but are not limited to disorders and diseases wherein the term “disorder” indicates a condition of the living individual that is associated to a functional abnormality of the body or of any of its parts, and the term “disease” indicates a condition of the living individual that impairs normal functioning of the body or of any of its parts and is typically manifested by distinguishing signs and symptoms. Exemplary conditions include but are not limited to injuries, disabilities, disorders (including mental and physical disorders), syndromes, infections, deviant behaviors of the individual and atypical variations of structure and functions of the body of an individual or parts thereof.

The wording “associated to” as used herein with reference to two items indicates a relation between the two items such that the occurrence of a first item is accompanied by the occurrence of the second item, which includes but is not limited to a cause-effect relation and sign/symptoms-disease relation.

Since recruitment of monocytes in the vascular wall represents a hallmark in atherosclerotic disease, we hypothesized that hypercholesterolemia and increased inflammation in atherosclerotic mice will result in increased thrombin cleavage of OPN, and that tcOPN could play an active role in atherosclerosis through the regulation of monocytes to the lesions. To test this hypothesis, we immunized apolipoprotein E knockout (ApoE^{-/-}) mice with a fused sequence, i.e., SLAYGLR (SEQ ID NO:1) fused with pan HLA DR-binding epitope (PADRE), to develop blocking antibodies against this site. PADRE is a carrier epitope in vaccines to induce strong antibody responses. We show that mice immunized with tcOPN-PADRE developed significantly less atherosclerosis and markedly reduced levels of tcOPN in plasma compared to the 13 amino acid pan DR epitope (PADRE) immunized control mice. Immunization with tcOPN-PADRE reduced atherosclerosis with >60% in descending aorta in mice given HFD for 8 weeks, and with >50% in mice given HFD for 15 weeks. Furthermore, immunization with tcOPN-PADRE reduced inflammatory monocytes, increased smooth muscle cell and collagen content in subvalvular plaques. Monocytes displayed increased migration towards tcOPN, which was inhibited by plasma from tcOPN-PADRE immunized mice.

As evidenced in Example 2, a study of 229 human carotid plaques, there were higher levels of tcOPN in lesions from diabetic patients that suffered from cardiovascular event symptoms prior to surgery compared to non-symptomatic diabetic patients or to patients without diabetic disease. High levels of tcOPN in plaques were also associated with increased incidence of postoperative death in the total cohort as well as to cardiovascular death and total death in T2D diabetic subjects during follow-up. The epitope that was used in immunizing mice against are believed to be relevant in human atherosclerotic plaques, especially in patients with diabetes.

The OPN or fragments, variants or peptidomimetics thereof can also be mixed or attached to adjuvants. The term “adjuvant” refers to a compound or mixture that enhances the immune response and/or promotes the proper rate of absorption following inoculation, and, as used herein, encompasses any uptake-facilitating agent. In one embodiment, the adjuvant is Montanide. Other non-limiting examples of adjuvants include, chemokines (e.g., defensins, HCC-1, HCC4, MCP-1, MCP-3, MCP4, MIP-1α, MIP-1β, MIP-1δ, MIP-3α, MIP-2, RANTES); other ligands of chemokine receptors (e.g., CCR1, CCR-2, CCR-5, CCR6, CXCR-1); cytokines (e.g., IL-1β, IL-2, IL-4, IL-5, IL-6, IL-7, IL-8, IL-10, IL-12, IL-13, IL-15, IL-17 (A-F), IL-18; IFNα, IFN-γ; TNF-α; GM-CSF); TGF)-β; FLT-3 ligand; CD40 ligand; other ligands of receptors for those cytokines; Th1 cytokines including, without limitation, IFN-γ, IL-2, IL-12, IL-18, and TNF; Th2 cytokines including, without limitation, IL-4, IL-5, IL-10, and IL-13; and Th17 cytokines including, without limitation, IL-17 (A through F), IL-23, TGF-β and IL-6; immunostimulatory CpG motifs in bacterial DNA or oligonucleotides; derivatives of lipopolysaccharides such as monophosphoryl lipid A (MPL); muramyl dipeptide (MDP) and derivatives thereof (e.g., murabutide, threonyl-MDP, muramyl tripeptide, N-acetyl-muramyl-L-threonyl-D-isoglutamine (thr-MDP); N-acetyl-nor-muramyl-L-alanyl-D-isoglutamine (CGP 11637, referred to as nor-MDP); N-acetylmuramyl-L-alanyl-D-isoglutaminyl-L-alani-ne-2-(1′-2′-dipalmitoyl-sn-glycero-3hydroxyphosphoryloxy)-ethylamine (CGP 19835A, referred to as MTP-PE)); MF59 (see Int'l Publication No. WO 90/14837); poly[di(carboxylatophenoxy)phosphazene] (PCPP polymer; Virus Research Institute, USA); RIBI (GSK), which contains three components extracted from bacteria, monophosphoryl lipid A, trehalose dimycolate and cell wall skeleton (MPL+TDM+CWS) in a 2% squalene/Tween 80 emulsion; OM-174 (a glucosamine disaccharide related to lipid A; OM Pharma SA, Meyrin, Switzerland); heat shock proteins and derivatives thereof; Leishmania homologs of elF4a and derivatives thereof; bacterial ADP-ribosylating exotoxins and derivatives thereof (e.g., genetic mutants, A and/or B subunit-containing fragments, chemically toxoided versions); chemical conjugates or genetic recombinants containing bacterial ADP-ribosylating exotoxins or derivatives thereof; C3d tandem array; lipid A and derivatives thereof (e.g., monophosphoryl or diphosphoryl lipid A, lipid A analogs, AGP, AS02, AS04, DC-Chol, Detox, OM-174); ISCOMS and saponins (e.g., Quil A, QS-21, Stimulon® (Cambridge Bioscience, Worcester, Mass.)); squalene; superantigens; or salts (e.g., aluminum hydroxide or phosphate, calcium phosphate). See also Nohria et al. Biotherapy, 7:261-269, 1994; Richards et al., in Vaccine Design, Eds. Powell et al ., Plenum Press, 1995; and Pashine et al., Nature Medicine, 11:S63-S68, 4/2005) for other useful adjuvants. Further examples of adjuvants can include the RIBI adjuvant system (Ribi Inc., Hamilton, Mont.), alum, mineral gels such as aluminum hydroxide gel, oil-in-water emulsions, water-in-oil emulsions such as, e.g., Freund's complete and incomplete adjuvants, Block co-polymer (CytRx, Atlanta Ga.), QS-21 (Cambridge Biotech Inc., Cambridge Mass.), and SAF-M (Chiron, Emeryville Calif.), AMPHIGEN® adjuvant, saponin, Quil A or other saponin fraction, monophosphoryl lipid A, and Avridine lipid-amine adjuvant, and METASTIM®. Other suitable adjuvants can include, for example, surface active substances such as lysolecithin, pluronic polyols, polyanions, peptides, oil or hydrocarbon emulsions, keyhole limpet hemocyanins, dinitrophenol, and others.

Osteopontin (OPN) is a highly inflammatory molecule, which is expressed during atherosclerosis. The OPN molecule contains several biological important sites including a cryptic SLAYGLR (SEQ ID NO:1) sequence, which is revealed in the N-terminal fragment after thrombin cleavage. The SLAYGLR (SEQ ID NO:1) sequence binds integrin α4β1, α4β7 and α9β1, which regulate immune cell functions, including adhesion and migration. However, the impact of this cryptic integrin binding site of thrombin cleaved OPN (tcOPN) in atherosclerotic lesion development is not known.

ApoE^{-/-} mice on high fat diet (HFD) displayed a more than 8 fold or 30 fold increased levels of tcOPN in plasma compared to chow-fed ApoE^{-/-} or wild type mice. To study the role of the cryptic site of tcOPN in atherosclerosis, we immunized ApoE^{-/-} mice with a SLAYGLR (SEQ ID NO:1) peptide to induce an antibody response against this site. The SLAYGLR (SEQ ID NO:1) peptide was synthesized together with pan HLA DR-binding epitope (PADRE), a carrier epitope in vaccines, which previously has been demonstrated to induce strong antibody responses. The mice were then kept on HFD for either 8 or 15 weeks to assess the effect of immunization on both early and advanced atherosclerosis. Mice immunized with tcOPN-PADRE developed a strong antibody response against tcOPN and had markedly reduced levels of tcOPN in plasma compared to mice immunized with PADRE alone. Immunization with tcOPN-PADRE reduced atherosclerosis with >60% in descending aorta in mice given HFD for 8 weeks, and with >50% in mice given HFD for 15 weeks. Furthermore, immunization with tcOPN-PADRE reduced inflammatory monocytes, increased smooth muscle cell and collagen content in subvalvular plaques. Monocytes displayed increased migration towards tcOPN, which was inhibited by plasma from tcOPN-PADRE immunized mice.

This study demonstrates that immunization-induced antibodies against the cryptic integrin binding site of tcOPN reduces atherosclerotic plaque development, decrease inflammatory monocytes and increase plaque stability.

Provided herein are fusion proteins, compositions, methods and systems that in several embodiments are suitable to be used for treatment or prevention of cardiovascular diseases.

Provided herein are compositions comprising osteopontin (OPN) or an antigenic fragment thereof and a protein carrier.

In exemplary embodiments, the protein carrier is bovine serum albumin (BSA), nonalbumin, edestin, exoprotein A from Pseudomonas aeruginosa, HC (hemocyanin from crab Paralithodes camtschatica), Helix promatia haemocyanin (HPH), human serum albumin (HSA), KTI (Kunits trypsin inhibitor from soybeans), keyhole limpet haemocyanin (KLH), LPH (haemocyanin from Limulus polyphemus), ovalbumin, Pam3Cys-Th, polylysine, porcine thyroglobulin (PTG), purified protein derivative (PPD), rabbit serum albumin (RSA), soybean trypsin inhibitor (STI), sunflower globulin (SFG), tetanus toxoid, diphtheria toxoid, PADRE (pan HLA DR-bindin epitope), Haemophilus influenza protein D, Neisseria meningitides outer membrane protein, flagellin, or CRM197.

In some embodiments, the OPN or the antigenic fragment thereof is fused to a protein carrier.

In one embodiment, the fragment of OPN is or includes the sequence SLAYGLR (SEQ ID NO:1) and the protein carrier is PADRE. In another embodiment, the fragment of OPN is or includes the sequence SVVYGLR (SEQ ID NO:2) and the protein carrier is PADRE.

In one embodiment of the composition, the antigen fragment consists of an amino acid sequence at least 85% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In another embodiment, the antigen fragment consists of an amino acid sequence at least 90% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In a further embodiment, the antigen fragment consists of an amino acid sequence at least 95% identical to the amino acid sequence SLAYGLR (SEQ ID NO:1) or to an epitope of OPN containing SLAYGLR (SEQ ID NO:1), such as GRGDSLAYGLR (SEQ ID NO:4) or VDVPNGRGDSLAYGLR (SEQ ID NO:5). In some embodiments, the fragment of OPN induces an antibody response.

Provided herein are methods for treating, inhibiting or reducing the severity of cardiovascular diseases in a subject in need thereof. The methods include administering to the subject, therapeutically effective amounts of compositions comprising osteopontin (OPN) or an antigenic fragment thereof and a protein carrier, as described herein. In one embodiment, the composition comprises a peptide having the sequence SLAYGLR (SEQ ID NO:1) and PADRE as the protein carrier. In some embodiments, the administration is performed via an oral, nasal, subcutaneous, or intramuscular route of administration.

Provided herein are methods for preventing cardiovascular diseases in a subject in need thereof. The methods include administering to the subject, therapeutically effective amounts of compositions comprising osteopontin (OPN) or an antigenic fragment thereof and a protein carrier, as described herein. In one embodiment, the composition comprises a peptide having the sequence SLAYGLR (SEQ ID NO:1), or SVVYGLR (SEQ ID NO:2), and PADRE as the protein carrier. In some embodiments, the administration is performed via an oral, nasal, subcutaneous, or intramuscular route of administration.

Further provided herein is an immunogenic composition comprising the compositions described herein together with an adjuvant and/or an excipient.

Further provided herein are vaccines comprising a peptide having the sequence SLAYGLR (SEQ ID NO:1), or SVVYGLR (SEQ ID NO:2), and PADRE as the protein carrier. In some aspects, the peptide having the sequence SLAYGLR (SEQ ID NO:1), or SVVYGLR (SEQ ID NO:2), is fused with PADRE.

In various embodiments, the compositions and vaccines according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The compositions and vaccines according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

In some embodiments of the methods described herein, the compositions described herein are administered sequentially or simultaneously with existing treatments for cardiovascular diseases. In some embodiments, the compositions and/or vaccines comprising the one or more peptides of OPN and a carrier protein (for example, OPN peptide having the sequence SLAYGLR (SEQ ID NO:1) and carrier protein PADRE) and optionally, an adjuvant as described herein are administrated to the subject 1-3 times per day or 1-7 times per week. In some embodiments, the compositions and/or vaccines comprising the one or more peptides of OPN and a carrier protein (for example, OPN peptide having the sequence SLAYGLR (SEQ ID NO:1) and carrier protein PADRE) and optionally, an adjuvant as described herein are administrated to the subject for 1-5 days, 1-5 weeks, 1-5 months, or 1-5 years. In some embodiments, the compositions and/or vaccines comprising the one or more peptides of OPN and a carrier protein (for example, OPN peptide having the sequence SLAYGLR (SEQ ID NO:1) and carrier protein PADRE) and optionally, an adjuvant as described herein are administrated to the subject in three doses per year, wherein the second dose is administered about 2-3 weeks, or about 3 weeks after the first dose and the third dose is administered about 5-6 weeks or about 6 weeks after the first dose. In some embodiments, the compositions and/or vaccines comprising the one or more peptides of OPN and a carrier protein (for example, OPN peptide having the sequence SLAYGLR (SEQ ID NO:1) and carrier protein PADRE) and optionally, an adjuvant as described herein are administrated to the subject in three doses per year, wherein the second dose is administered about 2-3 months, about 2 months, about 3 months or about 4 months after the first dose and the third dose is administered about 4-6 months, about 5-6 months, about 5 months or about 6 months after the first dose. In some embodiments, annual boosts may be administered to subject, wherein the boosts are administered at frequencies described herein, or monthly, every two months, every quarter, every six months, every year, or combination thereof, for up to 1-5 year, 5-10 year, 10-15 year or for the lifetime of the subject. Frequencies and duration of administration will be apparent to a person of skill in the art.

In various embodiments, the therapeutically or prophylactically effective amount of composition described herein for use with the methods described herein is any one or more of about 1-5 μg, 5-10 μg, 10-15 μg, 15-20 μg, 20-25 μg, 25-50 μg, 50-75 μg, 75-100 μg, 100-150 μg, 150-200 μg, 200-250 μg, 250-300 μg, 300-350 μg, 350-400 μg, 400-450 μg, 450-500 μg, 500-550 μg, 550-600 μg, 600-650 μg, 650-700 μg, 750-800 μg, 850-900 μg, 950-1000 μg, 5-500 μg, 50-500 μg, 100-500 μg, 200-500 μg, 300-500 μg, 400-500 μg or combinations thereof.

In various embodiments, the therapeutically or prophylactically effective amount of composition described herein for use with the methods described herein is any one or more of about 0.01 to 0.05 μg/kg/day, 0.05-0.1 μg/kg/day, 0.1 to 0.5 μg/kg/day, 0.5 to 5 μg/kg/day, 0.5 to 1 μg/kg/day, 1 to 5 μg/kg/day, 5 to 10 μg/kg/day, 10 to 20 μg/kg/day, 20 to 50 μg/kg/day, 50 to 100 μg/kg/day, 100 to 150 μg/kg/day, 150 to 200 μg/kg/day, 200 to 250 μg/kg/day, 250 to 300 μg/kg/day, 300 to 350 μg/kg/day, 350 to 400 μg/kg/day, 400 to 500 μg/kg/day, 500 to 600 μg/kg/day, 600 to 700 μg/kg/day, 700 to 800 μg/kg/day, 800 to 900 μg/kg/day, 900 to 1000 μg/kg/day, 0.01 to 0.05 mg/kg/day, 0.05-0.1 mg/kg/day, 0.1 to 0.5 mg/kg/day, 0.5 to 1 mg/kg/day, 1 to 5 mg/kg/day, 5 to 10 mg/kg/day, 10 to 15 mg/kg/day, 15 to 20 mg/kg/day, 20 to 50 mg/kg/day, 50 to 100 mg/kg/day, 100 to 200 mg/kg/day, 200 to 300 mg/kg/day, 300 to 400 mg/kg/day, 400 to 500 mg/kg/day, 500 to 600 mg/kg/day, 600 to 700 mg/kg/day, 700 to 800 mg/kg/day, 800 to 900 mg/kg/day, 900 to 1000 mg/kg/day or a combination thereof. Typical dosages can be in the ranges recommended by the manufacturer where known therapeutic compounds are used, and also as indicated to the skilled artisan by the in vitro responses or responses in animal models. Such dosages typically can be reduced by up to about an order of magnitude in concentration or amount without losing relevant biological activity. The actual dosage can depend upon the judgment of the physician, the condition of the patient, and the effectiveness of the therapeutic method based, for example, on the in vitro responsiveness of relevant cultured cells or histocultured tissue sample, such as biopsied malignant tumors, or the responses observed in the appropriate animal models. In various embodiments, the compositions described herein may be administered once a day (SID/QD), twice a day (BID), three times a day (TID), four times a day (QID), or more, so as to administer an effective amount to the subject, where the effective amount is any one or more of the doses described herein.

A method is also provided of treating, reducing the likelihood or severity of, or slowing the progression of cardiovascular death in a subject, including detecting an elevated level of thrombin-cleaved osteopontin (tcOPN) in a carotid plaque of the subject; and administering a therapeutically effective amount of a composition containing osteopontin (OPN) or an antigenic fragment thereof and a protein carrier to the subject. In various embodiments, the subject has diabetes, or type 2 diabetes. Generally, the elevated level of tcOPN is higher than an averaged value obtained from subjects without diabetes or from asymptomatic subjects.

In one embodiment, cardiovascular disease is atherosclerosis. In an exemplary embodiment, the atherosclerosis is accelerated atherosclerosis. Cardiovascular disease (CVD) is a class of diseases that involve the heart or blood vessels. Cardiovascular disease includes coronary artery diseases (CAD) such as angina and myocardial infarction (commonly known as a heart attack). Other CVDs include stroke, heart failure, hypertensive heart disease, rheumatic heart disease, cardiomyopathy, heart arrhythmia, congenital heart disease, valvular heart disease, carditis, aortic aneurysms, peripheral artery disease, thromboembolic disease, and venous thrombosis.

In various embodiments, the subject is selected from the group consisting of human, non-human primate, monkey, ape, dog, cat, cow, horse, rabbit, mouse and rat.

Pharmaceutical Composition

In various embodiments, the present invention provides a pharmaceutical composition comprising OPN or a fragment thereof and a protein carrier. In one embodiment, the composition comprises a peptide having the sequence SLAYGLR (SEQ ID NO:1), or SVVYGLR (SEQ ID NO:2), and PADRE as the protein carrier. The pharmaceutical composition includes The pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral or enteral. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art.

The pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins Pa., USA) (2000).

Before administration to patients, formulants may be added to the rAAV vector, the cell transfected with the rAAV vector, or the supernatant conditioned with the transfected cell. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

Another drug delivery system for increasing circulatory half-life is the liposome. Methods of preparing liposome delivery systems are discussed in Gabizon et al., Cancer Research (1982) 42:4734; Cafiso, Biochem Biophys Acta (1981) 649:129; and Szoka, Ann Rev Biophys Eng (1980) 9:467. Other drug delivery systems are known in the art and are described in, e.g., Poznansky et al., DRUG DELIVERY SYSTEMS (R. L. Juliano, ed., Oxford, N.Y. 1980), pp. 253-315; M. L. Poznansky, Pharm Revs (1984) 36:277.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention.

### Example 1 Mice study

### Experimental Methods

### Peptides and Proteins

An antibody response against peptides corresponding to the cryptic site exposed on tcOPN (SLAYGLR (SEQ ID NO:1)) was generated using synthetic oligonucleotides containing the sequence encoding the 13 amino acids of the PADRE epitope (AKFVAAWTLKAAA (SEQ ID NO:3)). OPN peptides of different length (11 amino acids, GRGDSLAYGLR (SEQ ID NO:4) (A) or 16 amino acids, VDVPNGRGDSLAYGLR (SEQ ID NO:5) (B)) were synthesized together with the PADRE sequence, resulting in two forms (one short, one long) of tcOPN-PADRE, i.e., AKFVAAWTLKAAAGRGDSLAYGLR (SEQ ID NO: 6) and AKFVAAWTLKAAAVDVPNGRGDSLAYGLR (SEQ ID NO: 7). PADRE peptide alone (SEQ ID NO:3) was used as control peptide. Alum was used as adjuvant and mixed together with peptide in 1:1 volume ratio. Peptide/alum mixture was freshly prepared prior to immunization.

Recombinant OPN and thrombin was purchased from R&D Systems. Thrombin cleavage was accomplished by incubation of OPN containing a C-terminal 6-His tag with thrombin at 37° C. during 4 hours. After cleavage, the C-terminal fragment of cleaved OPN was removed by His-Spin protein Miniprep (ZYMO RESEARCH).

### Animals

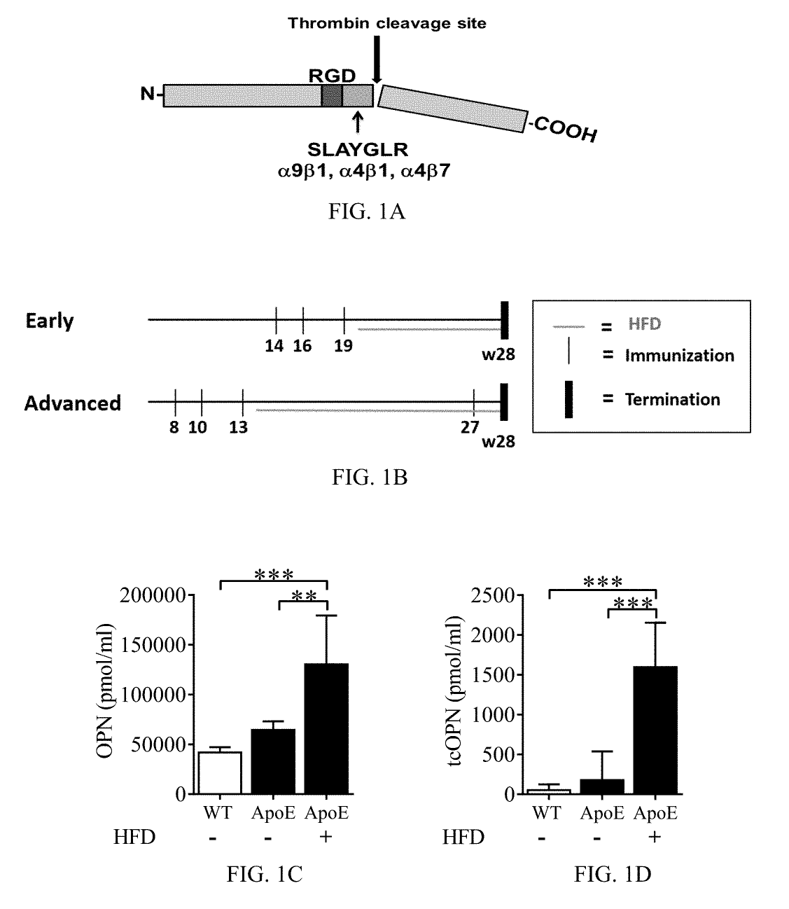

Female C57BL/6 Apoe^{-/-} mice (Jaxmice, USA) were immunized with tcOPN-PADRE or equal amounts of PADRE peptide using Alum (Pierce) as adjuvant, followed by two booster injections after two and five weeks (FIG. 1B). During the advanced study all mice received a final booster one weak prior to termination in order to analyze the effect on immune cells (FIG. 1B). Mice were fed a high-fat diet (HFD), 21% cocoa fat, 0.15% cholesterol, starting one week after the last booster injection. For early assessment of atherosclerosis mice were kept on HFD during 8 weeks before sacrifice. Mice that were allowed to develop a more advanced stage of atherosclerosis were kept on HFD during 15 weeks (FIG. 1B). For early atherosclerosis assessment two different lengths of immunized tcOPN-PADRE peptides (SEQ ID Nos: 6 and 7) were evaluated, while for advanced atherosclerosis assessment mice were immunized with shorter tcOPN-PADRE (SEQ ID No: 6) which was based on a tcOPN of 11 amino acids (SEQ ID NO: 4). Tissue was preserved as described previously. All animal experiments were approved by the Animal Care and Use Committee. Cytokine levels for IL-1β, IL-2, IL-4, IL-5, IL-6, IL-10, IL-13, IL-17, IL-12(p70), IFNγ, TNFα and MCP-1 were analyzed in plasma using Bioplex multiplex technology (Bio-Rad) according to manufacturer's protocol. Plasma levels for OPN and tcOPN were analyzed by ELISA according to manufacturer's protocol (IBL International). Plasma cholesterol was quantified colorimetrically using Infinity Cholesterol (ThermoTrace).

### Staining of the Descending Aorta

En face preparations of the descending aorta were washed in distilled water, dipped in 78% methanol, and stained for 40 min in 0.2% Oil-Red-0 dissolved in 78% methanol, 0.2 mol/L NaOH. Stained area and total aortic areas were quantified blinded by microscopy and computer aided morphometry (Olympus Micro Image, Hamburg, Germany and BioPix iQ 2.0, Göteborg, Sweden).

Analysis of Plaque Macrophage, α-actin, collagen and Ly6c Content

The heart was embedded in OCT (optimal cutting temperature; Tissue-Tek, Zoeterwoulde). Frozen sections, 10 μm of subvalvular lesions were collected. For macrophage, α-actin and Ly6c content, subvalvular plaques were stained with MOMA-2 antibody (BMA Biomedicals, Switzerland), smooth muscle α-actin (α Actin Smooth Muscle antibody, clone 1A4, Sigma, A2547) and anti-Ly6c (Mouse Ly-6C BD Pharmingen, 557359), respectively, using rabbit or rat immunoglobulins (Dako, Solna, Sweden) as negative controls. DAB detection kit was used for color development (Vector Laboratories, CA) and the sections were counterstained in haematoxylin. For assessment of the collagen content of the plaques, subvalvular sections were stained with Van Gieson Solution Acid Fuchsin (Sigma-Aldrich). All stainings were quantified with BioPix iQ 2.0.

### Antibody ELISA

tcOPN-PADRE peptide, PADRE peptide, tcOPN protein or OPN protein was used for coating (20 μg/mL of each in PBS) microtiter plates (Nunc MaxiSorp, Nunc) over night at 4° C. Coated plates were washed with PBS with 0.05% Tween-20 and thereafter blocked with SuperBlock in Tris-buffered saline (TBS, Pierce) during 30 minutes at room temperature (RT) followed by an incubation of mouse plasma diluted 1:100 or 1:1000 in PBS-0.05% Tween-20 for 2 hours at RT. After rinsing, bound antibodies were detected using biotinylated goat anti-mouse IgGla or IgG2 antibodies (Jackson ImmunoResearch) that were incubated for 2 hours at RT. After washing, alkaline phosphatase-conjugated streptavidin (Sigma) was added and color reaction was developed using phosphatase substrate kit (Pierce). The absorbance (405 nm) was measured after 1 hour of incubation at room temperature. Mean values were calculated after subtraction of background absorbance.

### Splenocyte Isolation

Single-cell suspensions of splenocytes were prepared by pressing spleens through a 70-μm cell strainer (BD Falcon, Franklin Lakes, N.J.). Erythrocytes were removed using red blood cell lysing buffer (Sigma-Aldrich). Cells were cultured in culture medium containing 10% heat-inactivated FCS, 1 mmol/L sodium pyruvate, 10 mmol/L Hepes, 50 U of penicillin, 50 μg/mL streptomycin, 0.05 mmol/L β-mercaptoethanol, and 2 mmol/L L-glutamine (RPMI 1640, GIBCO, Paisley, UK) in 48-well plates (Corning).

### Flow Cytometry Analysis

Shortly, for detection of monocytes 25 μl of blood cells or 5×10⁵ spleen cells were stained with pacific blue (PB) anti-mouse CD11b (Biolegend), phycoerythrin (PE) anti integrin α9 (R&D Systems), PE/Cy7 anti-mouse Ly-6C (Biolegend) and FITC anti-mouse CD49d (Biolegend). Regulatory T-cells were analyzed in splenocytes, cells were stained with PE/Cy7 CD3 (Biolegend), PB CD4 (Biolegend) and APC CD25 (Biolegend) followed by permeabilization and staining for intracellular PE FoxP3 (Biolegend). For the detection of IFN-γ and IL-5 positive T-cells cells, splenocytes (5×10⁵ cells/cell culture well) were first incubated with phorbol 12-myristate 13-acetate (PMA; 10 ng), ionomycin (0.2 μg), and brefeldin A (1 μg, all from Sigma) for 4 hours at 37° C. Stimulated cells were then stained for PE/Cy7 CD3 (Biolegend) and PB CD4 (Biolegend). Cells were thereafter permeabilized and stained with PE anti-IFNγ (Biolegend) or APC anti-IL-5 (Biolegend). Measurements were performed using a CyAn ADP flow cytometer (Beckman Coulter, Brea, Calif.) and analyzed with Summit software (Tree Star, Ashland, Oreg.). Mononuclear leukocytes were gated from the forward scatter (FSC)/side scatter (SSC). Single stained samples were used to correct for fluorescence spillover in multicolor analyses, and gate boundaries were set by fluorescence-minus-one controls.

### Migration Assay

Monocyte cell migration was quantified in 24-well transwell inserts with polycarbonate filters (8-μm pore size) (Corning Costar). THP-1 monocytes (0.5×10⁴ in 100 μl of RPMI 1640 medium) or murine blood cells from ApoE^{-/-} mice (25 μl in 75 μl of RPMI 1640 medium) were added to the upper chamber of the insert. The lower chamber contained 600 μl of RPMI 1640 with or without 10ng/ml tcOPN or OPN protein. The plates were incubated at 37° C. in 5% CO₂ during 2 hours and migrated cells in the lower chamber were counted using flow cytometry. Blood monocytes were characterized with the monocyte FACS panel described above. In blocking experiment, tcOPN protein were pre-incubated with plasma (dilution 1:100) pooled from PADRE ctrl mice or tcOPN-PADRE mice over night at 4° C. prior to the migration experiment.

### Peptide-Integrin Binding Assay

Integrin α9β1, α4β1 and α4β7 (R&D Systems) was used for coating (20 μg/mL of each in PBS) microtiter plates (Nunc MaxiSorp, Nunc) over night at 4° C. SLAYGLR (SEQ ID NO:1) peptide (20 μg/mL) was pre-incubated with either PBS or with plasma from PADRE or tcOPN-PADRE immunized mice (diluted 1:100 in PBS with 0.05% Tween-20) during 90 min on shaker. Coated plates were washed with PBS with 0.05% Tween-20 and thereafter blocked with SuperBlock in Tris-buffered saline (TBS, Pierce) during 30 minutes at room temperature (RT), followed by an incubation with SLAYGLR (SEQ ID NO:1) peptides with or without plasma from PADRE or tcOPN-PADRE immunized mice during 2 hours at RT. After rinsing, bound peptide were detected using anti-Osteopontin N-Half (34E3) antibodies (IBL International) that were incubated for 1 hour followed by washing and HRP anti Mouse (Dako, Solna, Sweden) during 30 min. A color reaction was developed using TMB substrate kit (Pierce). The absorbance was measured at 450 nm after 1 hour of incubation at room temperature. Mean values were calculated after subtraction of background absorbance. Binding of peptides without pre-incubation were used as control and values set to 100%.

### Statistical Analysis

Statistical analyses were performed with GraphPad version 6.04. Values are presented as mean ±SD, if not otherwise indicated. For skewed variables the non-parametric Mann-Whitney-test was used for comparisons of data. Statistical significance was considered at the level *P<0.05, **P<0.01 or ***P<0.001.

### High Fat Diet Increased the Proteolytic Digestion of OPN by Thrombin

Hypercholesterolemia and atherosclerosis are associated with increased thrombin generation. In order to test the hypothesis that the proteolytic environment in hypercholesterolemia will cause increased cleavage of OPN by thrombin and thereby expose the cryptic integrin binding site on OPN, we analyzed plasma from atherosclerotic ApoE^{-/-} mice kept on HFD. The result demonstrates that both full-length OPN and tcOPN were increased in plasma from ApoE^{-/-} mice on HFD. Full-length OPN was increased two-fold compared to plasma from ApoE^{-/-} mice on normal chow and three-fold compared to wild type mice (FIG. 1C). tcOPN was increased with an almost nine-fold compared to ApoE^{-/-} mice on normal chow and a thirty-fold compared to wild type mice (FIG. 1D).

Immunization of ApoE^{-/-} Mice with tcOPN Resulted in High IgG Titers Against tcOPN and Reduces Levels of tcOPN in Plasma.

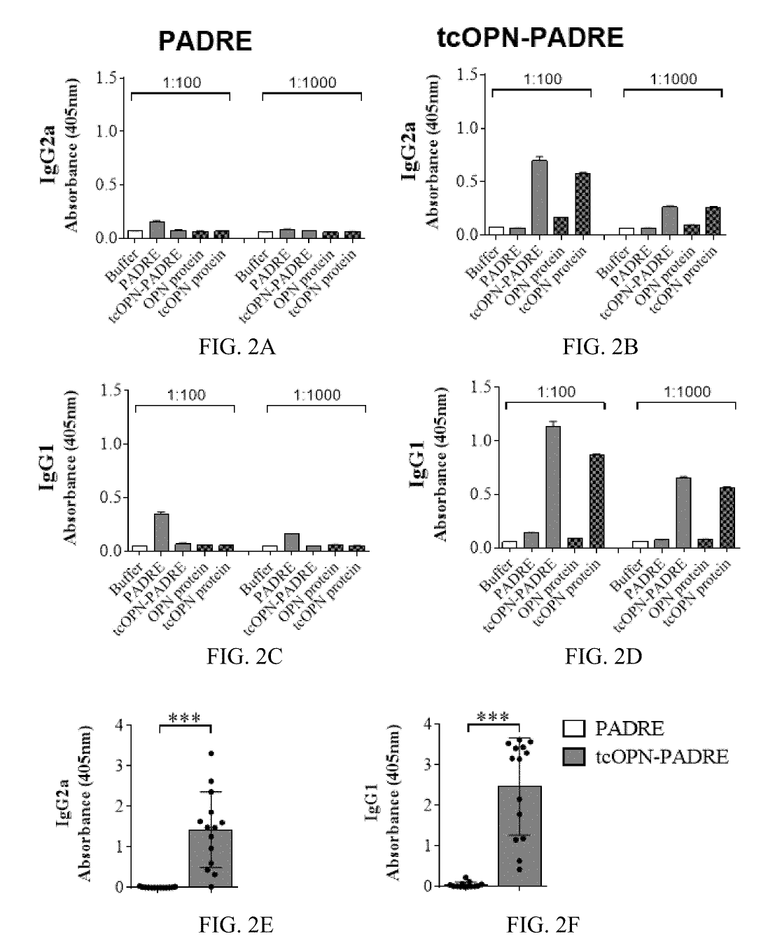

To study the role of the cryptic site of tcOPN in atherosclerosis we immunized ApoE^{-/-} mice with a peptide containing the SLAYGLR (SEQ ID NO:1) sequence to induce an antibody response. An 11 amino-acid or 16 amino acid peptide containing the SLAYGLR (SEQ ID NO:1) sequence was synthesized together with PADRE, resulting in tcOPN-PADRE (SEQ ID NO: 6 or SEQ ID NO: 7, respectively), which previously has been demonstrated to induce strong antibody responses against coupled peptides. To study the effect on both early and advanced atherosclerosis, the mice were given HFD for either 8 or 15 weeks after immunization. There were no detectable IgG levels against tcOPN in ApoE^{-/-} mice prior to immunization (data not shown). Mice immunized with the cryptic integrin binding site of tcOPN coupled to PADRE (from now on termed tcOPN-PADRE), displayed high antibody titers against the tcOPN-PADRE peptide (FIG. 2B, 2D, 2E and 2F). Furthermore, the antibodies detected thrombin cleaved OPN protein, but not full-length OPN. The IgG response was primarily of the IgG1 type, suggesting a predominance of Th2 immune responses (FIG. 2B, 2D, 2F). Control mice immunized with PADRE alone did not develop antibodies against tcOPN-PADRE (FIG. 2A, 2C), but displayed low levels of antibodies recognizing PADRE peptide. Importantly, the immunization was associated with a significant decrease in circulating levels of tcOPN, whereas levels of full-length OPN was not affected (FIG. 2G-2H).

Immunization with tcOPN-PADRE Reduced Inflammatory Monocytes

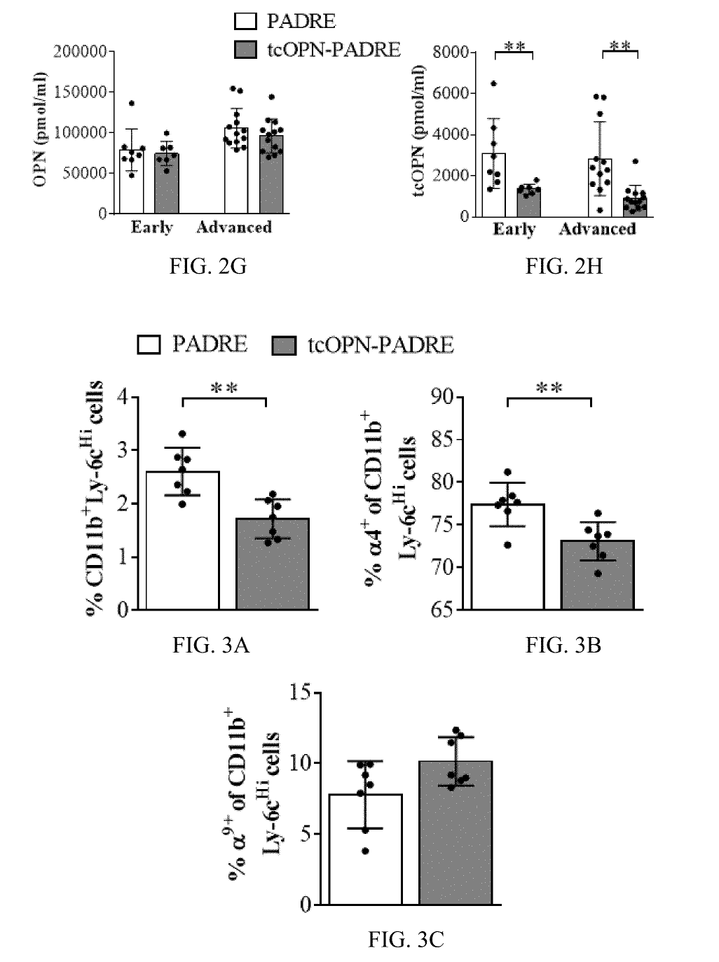

We then examined the effect of immunization against tcOPN-PADRE on immune cells. Flow cytometry analysis of splenocytes from tcOPN-PADRE immunized mice sacrificed after 8 weeks of HFD showed no differences in the fraction of Th1 (CD3⁺IFNγ⁺), Th2 (CD3⁺IL-5⁺) or Treg (CD3⁺CD4⁺CD25⁺FoxP3³⁰ ) cells compared to PADRE immunized control mice. A similar result was obtained in mice after 15 weeks on HFD, but with an additional increase in Treg cells present in tcOPN-PADRE immunized mice. No differences in plasma Th1-, Th2-, Th17-, or Treg-associated cytokines could be detected between immunized mice in either early or advanced atherosclerosis (Tables 3 and 4). tcOPN-PADRE immunized mice sacrificed after 8 weeks of HFD displayed lower plasma levels of monocyte chemoattractant protein-1 (MCP-1) (Table 3). Interestingly, immunization with tcOPN-PADRE resulted in significantly less inflammatory (Ly6C^{Hi}) monocytes (FIG. 3A). Furthermore, Ly6c^{Hi} monocytes from tcOPN-PADRE immunized mice expressed lower levels of α4 integrin (FIG. 3B), whereas no difference was detected in α9 integrin expression compared to PADRE immunized mice (FIG. 3C).

TABLE 3
Plasma cytokine profile 8 week HFD. Plasma levels
(pg/ml) of cytokines expressed as mean ± SD.
A significant difference between PADRE and tcOPN-
PADRE is indicated with *. N.D. denotes non-detectable.

| | | tcOPN- |
|---|---|---|
| | PADRE | PADRE |
| IL-2 | 45 ± 15 | 52 ± 0 |
| IL-4 | N.D | N.D |
| IL-5 | 111 ± 56 | 162 ± 173 |
| IL-6 | 33 ± 35 | 32 ± 20 |
| IL-10 | 77 ± 32 | 115 ± 25 |
| IL-13 | 234 ± 129 | 384 ± 274 |
| IL-17 | 162 ± 62 | 193 ± 108 |
| TNFα | 26 ± 0 | 49 ± 17 |
| IFNγ | 312 ± 225 | 378 ± 224 |
| MCP-1 | 835 ± 187 | 585 ± 235 * |

TABLE 4
Plasma cytokine profile 15 week HFD.
Mean ± SD plasma pg/ml values

| | | tcOPN- |
|---|---|---|
| | Control | PADRE |
| IL-2 | 43 ± 21 | 46 ± 22 |
| IL-4 | 21 ± 7 | 23 ± 10 |
| IL-5 | 110 ± 32 | 127 ± 48 |
| IL-6 | 28 ± 11 | 28 ± 13 |
| IL-10 | 172 ± 64 | 157 ± 68 |
| IL-13 | 1153 ± 439 | 1316 ± 673 |
| IL-17 | 148 ± 27 | 154 ± 35 |
| TNFα | 712 ± 249 | 753 ± 316 |
| IFNγ | 48 ± 24 | 54 ± 34 |
| MCP-1 | 703 ± 386 | 633 ± 232 |

Immunization with tcOPN-PADRE Reduced the Atherosclerotic Development in ApoE^{-/-} Mice

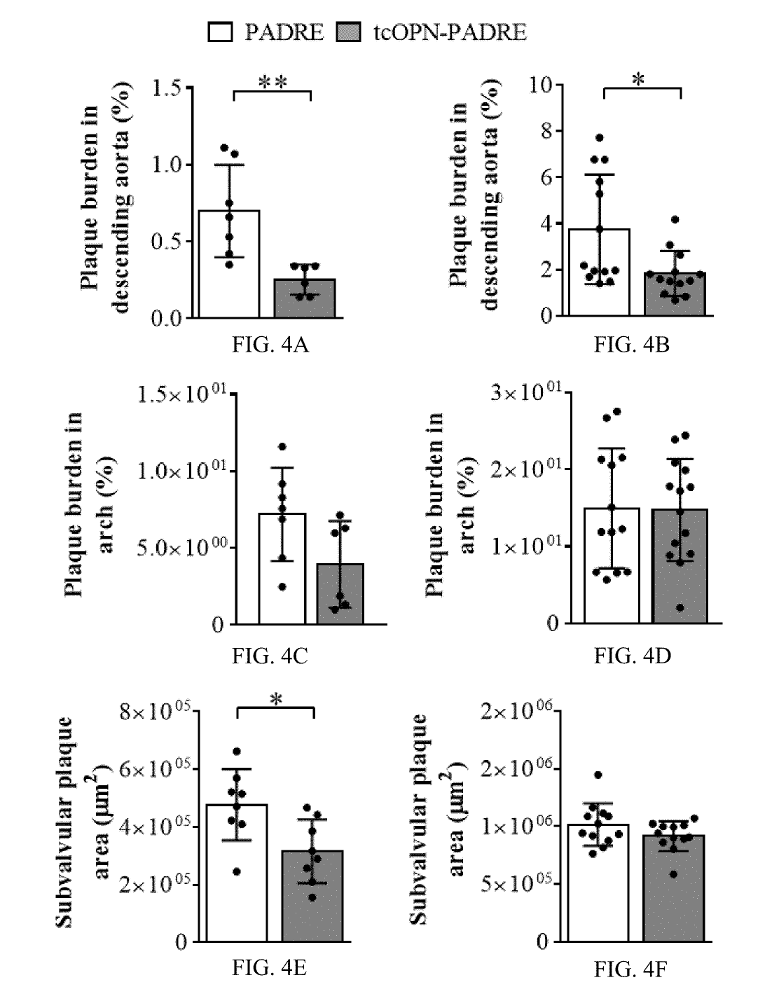

Next we investigated the effect of immunization against the cryptic integrin binding sites of tcOPN on atherosclerosis. Immunization with tcOPN-PADRE reduced plaque area in the descending aorta with >60% in early atherosclerosis (FIG. 4A) and >50% in advanced atherosclerosis (FIG. 4B). The effect in aortic arch was less evident and only a trend to reduced plaque area was detected in the early atherosclerosis study (P=0.051, FIG. 4C), while there was no effect in the advanced atherosclerosis (FIG. 4D). Immunization also reduced plaque size in subvalvular lesions in mice kept on HFD during 8 weeks compared to control immunized mice (FIG. 4E). There was no difference in plaque size in subvalvular lesions in immunized mice kept on HFD during 15 weeks (FIG. 4F). Immunization had no effect on plasma cholesterol levels in the study of early arthrosclerosis (Table 5). However, tcOPN-PADRE immunized mice fed a HFD for 15 weeks displayed a small increase in cholesterol levels (Table 5).

TABLE 5
Plasma cholesterol levels.

| | | tcOPN- |
|---|---|---|
| | PADRE | PADRE |
| Cholesterol (mg/dL) early atherosclerosis | 772 ± 154 | 683 ± 60 |
| Cholesterol (mg/dL) advanced atherosclerosis | 647 ± 143 | 765 ± 113 * |

Plasma cholesterol levels expressed as mean ± SD. A significant difference between PADRE immunized mice and tcOPN-PADRE immunized mice is indicated with *.

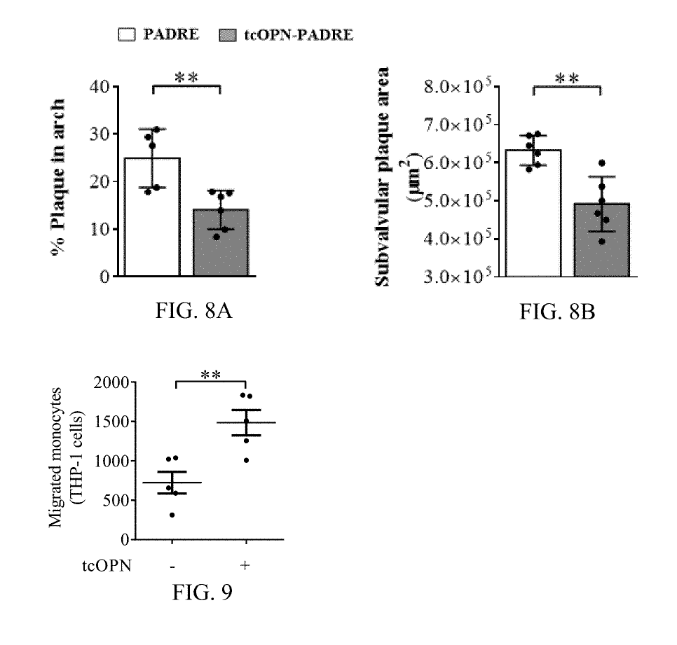

Since the tcOPN-PADRE peptide used in early atherosclerosis study (29 amino acids, SEQ ID NO: 7) contained an additional five amino acids (amino terminal of the cryptic site) and therefore was slightly longer than the tcOPN-PADRE peptide used in the advanced atherosclerosis study (24 amino acids, SEQ ID NO: 6), we performed an additional study on early atherosclerosis. In this study, we immunized ApoE^{-/-} mice with the short tcOPN-PADRE peptide (31 amino acids) and kept the mice on HFD for 8 weeks. Immunization using the short tcOPN-PADRE peptide recapitulated the difference seen in subvalvular plaque area (FIG. 8B) and displayed a smaller plaque area in the aortic arch in en face preparations (FIG. 8A). In agreement with the study with shorter peptide, plasma cholesterol levels did not differ between PADRE and tcOPN-PADRE immunized mice (813±109 vs 772±137 mg/dL).

Plaques from tcOPN-PADRE Immunized Mice are Smaller and Have a More Stable Phenotype.

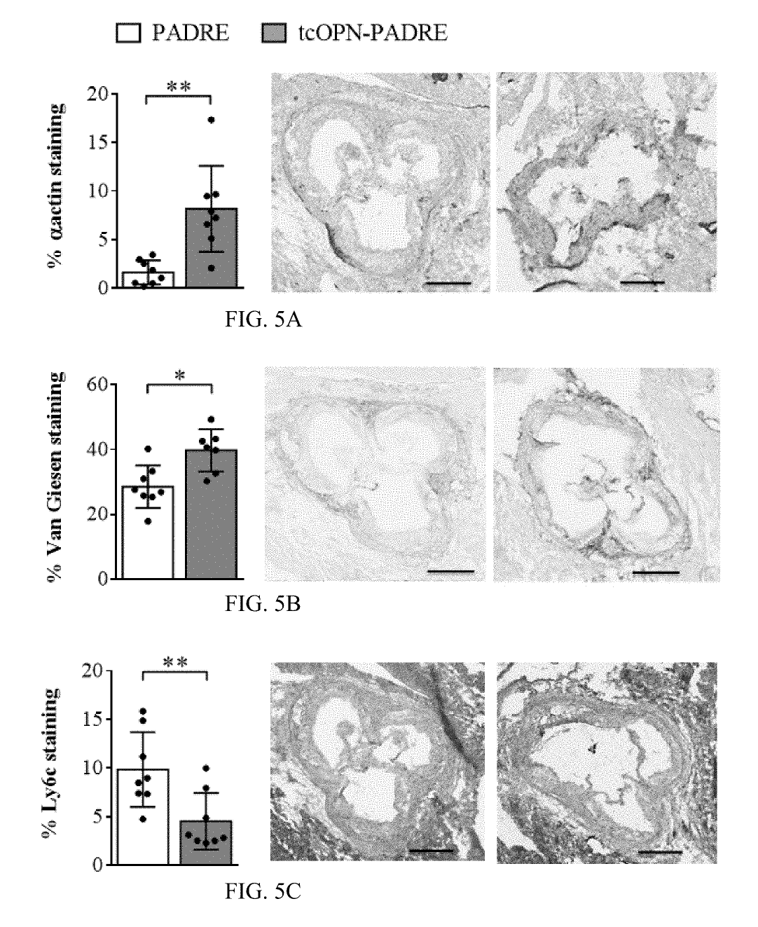

To study the effect of immunization with the cryptic site of tcOPN on atherosclerotic plaque composition, we analyzed subvalvular plaques from mice kept on HFD for eight weeks. Plaques from tcOPN-PADRE immunized mice contained significantly more vascular smooth muscle cells (FIG. 5A) and collagen (FIG. 5B). It has previously been demonstrated that monocytes migrate in response to tcOPN, and we therefore analyzed the content of inflammatory monocytes in the lesions. Plaques from tcOPN-PADRE immunized mice contained significantly reduced amounts of inflammatory Ly6c⁺ monocytes compared to PADRE immunized mice (FIG. 5C). There was no difference in the amount of macrophages, assessed by MOMA staining (data not shown).

Monocytes Migrate to the Cryptic Integrin Binding Site on tcOPN

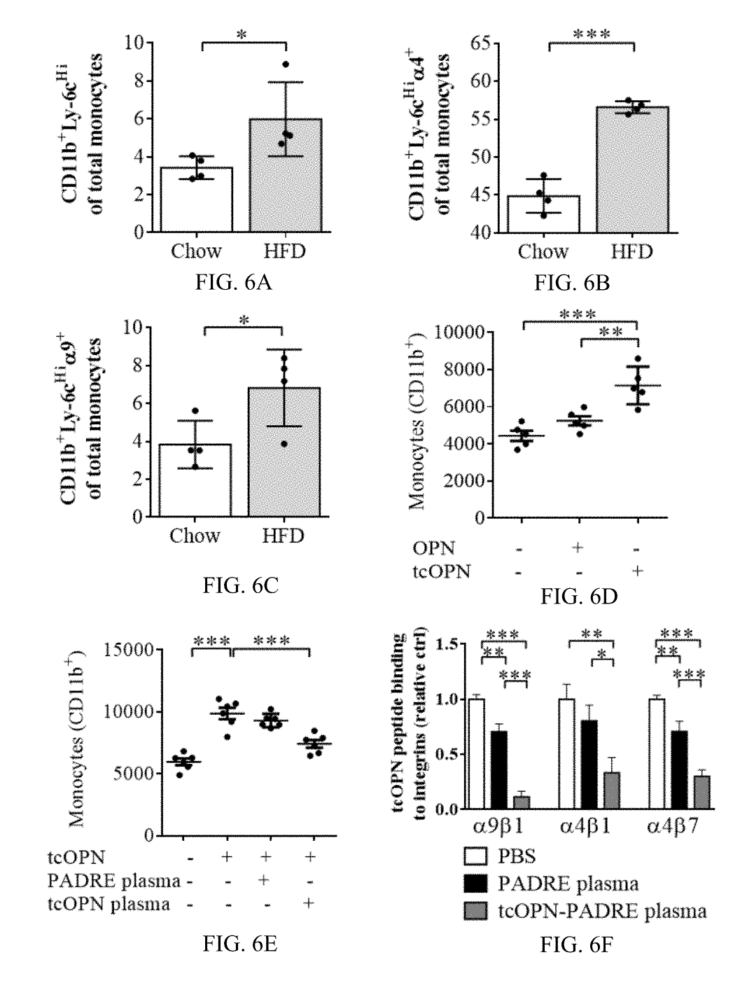

To examine whether inflammatory monocytes express integrins, which could interact with the cryptic integrin binding site on tcOPN, we obtained monocytes from ApoE^{-/-} mice given HFD or chow diet. Spleens from mice given HFD mice contained significantly more inflammatory Ly-6C^{Hi} monocytes (CD11b⁺Ly6C^{Hi}) compared to mice on chow diet (FIG. 6A). Furthermore, Ly6C^{Hi} monocytes from HFD mice expressed more α4 and α9 integrins compared to chow fed mice (FIGS. 6B and 6C).

It has previously been reported that monocytes migrate in response to tcOPN and plays an important role during the pathogenesis of rheumatoid arthritis. Since recruitment of monocytes to the vascular wall is one of the earliest steps in atherosclerotic disease, we investigated the effect of immunization-induced antibodies on monocyte migration. First we analyzed migration of monocytes using a transwell system. Blood monocytes from ApoE^{-/-} mice (CD11b⁺) (FIG. 6D) and human monocyte THP-1 cells displayed increased migration towards tcOPN compared to buffer alone or full-length OPN protein. To evaluate the blocking effect of antibodies from immunized mice, tcOPN protein was pre-incubated with plasma from PADRE or tcOPN-PADRE immunized mice. The migratory effect of monocytes towards tcOPN was reduced with more than 60% by addition of plasma from tcOPN-PADRE immunized mice, while plasma from PADRE immunized mice had no effect (FIG. 6E). The cryptic binding site of OPN contains integrin binding sequences for the α9β1, α4β1 and α4β7 integrin. To examine the blocking effect of antibodies from immunized mice on specific integrin interactions, a peptide corresponding to the peptide-binding sites on tcOPN was pre-incubated with plasma from PADRE or tcOPN-PADRE immunized mice. Peptide to integrin binding was reduced with more than 80% for α9β, 55% for α4β1 and 55% for the α4β7 integrin by addition of plasma from tcOPN-PADRE compared to PADRE immunized mice (FIG. 6F). Taken together these results indicated that tcOPN has the potential to mediate infiltration of inflammatory monocytes induced by HFD, and that this could be blocked by immunization against the cryptic integrin binding site of tcOPN.

In this study, we evaluated the effect of tcOPN in the development of experimental atherosclerosis. First, we demonstrated that hypercholesterolemia increases thrombin cleavage of OPN with almost a tenfold compared to mice without HFD. We then used a peptide-based vaccine approach to induce antibodies against the cryptic integrin binding site exposed on tcOPN. We found that immunizing mice with the cryptic integrin binding epitope reduced tcOPN protein in the circulation and markedly diminished the development of atherosclerosis in ApoE-deficient mice. The effect on plaque development was accompanied with a more stable plaque phenotype, where plaques from immunized mice showed an increase in collagen and smooth muscle content as well as reduced levels of the inflammatory marker Lytic. We could also demonstrate that monocytes migrated towards the cryptic tcOPN integrin binding epitope, which was inhibited by plasma from tcOPN-PADRE immunized mice. The result from the present study suggests that the cryptic integrin binding site exposed on tcOPN plays an important role in the development of atherosclerotic plaques in mice.

Increased OPN expression is frequently found in inflammatory conditions including atherosclerosis. Multiple studies have focused on the involvement of OPN in atherosclerosis and substantial evidence show that OPN plays an important role in the development and pathogenesis of the disease, both in animal models as well as in human disease. The cryptic integrin binding site exposed on tcOPN is less studied. In acute injury this site seems to have a protective role, as shown by Doyle et al where intranasal administration of a peptide that mimics the exposed site on tcOPN induces neuroprotection in a model of ischemic brain injury in mice. However both thrombin and tcOPN are increased during atherosclerosis and hyperlipidemia, and it is possible that during chronic inflammatory conditions, including atherosclerosis, tcOPN may be constantly exposed which could worsen the inflammatory state. The functional role of tcOPN in atherosclerosis has not been investigated, but several studies indicate that tcOPN is more potent in terms of activating inflammation compared to full-length OPN. It has been shown that several cell types, including inflammatory cells adhere specifically to tcOPN. Furthermore, Lund et al has demonstrated that integrin α4 and α9 on macrophages interact with OPN through the cryptic tcOPN domain, which regulate macrophage migration, survival, and accumulation. Yamamoto et al showed that monocyte migration towards tcOPN could be almost completely inhibited by using a blocking antibody against the tcOPN motif, resulting in diminished rheumatoid arthritis. Studies performed on human atherosclerotic plaques indicate that tcOPN are associated with increased inflammation and cardiovascular symptoms. Wolak et al found that tcOPN, rather than full-length OPN, associates with carotid plaque inflammation in hypertensive patients. In a recent study OPN and tcOPN levels were analyzed in carotid plaques in patients with or without statin treatment. The authors found that statin treatment was associated with more stable plaques and a significantly lower plaque content of tcOPN compared to non-statin treated patients. Unlike plaque data, measurement of tcOPN in human plasma has so far failed to detect a connection to atherosclerosis. Yan et al measured OPN and tcOPN in plasma from patients with type 2 diabetes mellitus and investigated their association to coronary artery disease, however only an association between full-length OPN and coronary atherosclerosis was found. The study from Yan et al focuses on patients with type 2 diabetes, and further clinical studies investigating tcOPN in human plasma are needed to evaluate the possible use of tcOPN as a marker of cardiovascular diseases. Taken together, these findings indicate that tcOPN could play an important role in atherosclerotic plaque development, at least during the inflammatory phase. In the current study, we show that by inducing antibodies against the cryptic integrin binding site of tcOPN in atherosclerotic prone mice, atherosclerotic lesion development is reduced and a more stable plaque phenotype is induced. Our results strengthen previous findings in human plaque material and demonstrate that atherosclerotic mice can be successfully treated to reduce the inflammatory effects of this epitope.

Besides the result on reduced development of atherosclerosis after tcOPN immunization in our study, we found a decreased fraction of Ly-6C^{hi} monocytes in the circulation (FIG. 3A-3C), lower Lytic staining in plaque tissue and (FIG. 5A-5C), and reduced levels of the monocyte associated MCP-1 cytokine in plasma (Table 3). Together these results point to a connection between inflammatory monocytes and tcOPN. It is well established that hypercholesterolemia leads to the development of atherosclerosis and that key steps in this process are monocyte recruitment and migration into atherosclerotic lesions. Inside the vascular wall monocytes differentiate to macrophages and cause increased inflammation, which could destabilize the plaque. It has also been shown that Ly-6C^{hi} monocytes are associated with increased inflammation and atherosclerosis. Monocytes express integrin α4 and α9, which binding sites are exposed on tcOPN. Consequently, it is likely that increased levels of tcOPN results in activation and migration of inflammatory monocytes. Results from the present study demonstrate that hypercholesterolemia increases both the level of tcOPN and Ly-6C^{hi} monocytes in the circulation (FIGS. 3A-3C). We also demonstrate that monocytes have the capacity to migrate towards tcOPN in vitro and that plasma from immunized mice could inhibit the migratory effect of tcOPN on monocytes (FIG. 6A-6F). These results are in accordance by Yamamoto et al, who showed that inflammatory cell infiltration in arthritic joints was inhibited by treating with an antibody against tcOPN. Collectively, these data indicates that tcOPN attracts inflammatory monocytes in atherosclerosis and blocking this epitope inhibits atherosclerotic lesion development.

In our study on advanced atherosclerosis we also found an increased fraction of Tregs in tcOPN-PADRE immunized animals. Regulatory T-cell populations are immunosuppressive cells that have an important role in maintaining self-tolerance and protection against autoimmunity. In atherosclerosis it has been demonstrated that Tregs inhibit the development of atherosclerosis in mice. Thus, the increase in Tregs in the current study could be part of the mechanism explaining the athero-protection induced by immunization. However, since Tregs has important immunosuppressive functions on other T-cell populations and our results show no effect on Th1 or Th2 cells or on any of the measured circulating T-cell related cytokines (Table 4) it is unlikely that the increased fraction of Tregs could be solely responsible for the reduced level of atherosclerosis after immunization. Moreover, the fraction of Tregs or T-cell related cytokines were not altered in the study of early atherosclerosis, where the strongest inhibition of atherosclerosis was seen (FIG. 3A-3C). This further supports that Tregs are not likely to be the mediator of the athero-protection induced by tcOPN-PADRE immunization.

The level of tcOPN in mice was reduced to about 56-68% in this study. tcOPN are likely to be present to some extent in plasma and our model therefore could have a better biological relevance to study the impact that an increase of tcOPN would exert on atherosclerosis, compared to a knockout model.

This study demonstrates that HFD induce the proteolytic cleavage of OPN by thrombin. The exposed cryptic site on tcOPN has the ability to attract immune cells including inflammatory monocytes. Introducing antibodies specific for the exposed integrin binding site on tcOPN, significantly suppressed atherosclerotic development in mice.

### Example 2. Content of Thrombin Cleaved Osteopontin in Carotid Atherosclerotic Plaques was Associated with Cardiovascular Death in type II diabetes (T2D) Patients

### Background

Human carotid plaques contain OPN, whose amount has been associated with inflammation, instability and to future cardiovascular events. OPN is modulated by thrombin, an enzyme expressed during inflammatory conditions like atherosclerosis and diabetes. The thrombin cleaved form of OPN (tcOPN) in human atherosclerosis has only been sparsely studied. The aim of this study was to analyze if tcOPN were associated with instability of human carotid plaques and if tcOPN has a predictive value for future cardiovascular events.

### Overall Methodology

In total, 229 carotid plaques obtained by endarterectomy were included in the study. Participants were monitored for outcomes, including cardiovascular events and death. Levels of full length as well as tcOPN were analyzed by ELISA, and the levels were examined to symptoms prior to surgery and to clinical outcome during follow-up period.

### Patient Characteristics

Two hundred twenty-nine, 229, human carotid plaques were collected at carotid endarterectomy from 89 subjects with DM II and 140 subjects without DM II (Table 6) and were included in the Coronary Plaque Imaging Project (CPIP). Three patients, all asymptomatic non-diabetics, underwent a second endarterectomy. The indications for surgery were plaques associated with ipsilateral symptoms (transitory ischemic attack, stroke or amaurosis fugax) and stenosis, measured by duplex, >70% (n=117) or plaques not associated with symptoms and stenosis >80% (n=112). All plaques from symptomatic patients were removed within 32 days after symptoms. Informed consent was given by each patient. The study was approved by the

Regional ethics committee in Lund (approval no 472/2005) and conducted in agreement with the Declaration of Helsinki. All patients were preoperatively assessed by a neurologist. We evaluated cardiovascular risk factors, namely hypertension (systolic blood pressure >140 mm Hg), diabetes and current smoking and took into account the use of medications (anti-hypertensive drugs, diabetes treatment and statins). We measured fasting lipoproteins (total cholesterol, HDL cholesterol, LDL cholesterol and triglycerides). Blood samples were collected one day before endarterectomy. Informed consent was given by each patient. The study was approved by the local ethical committee.

TABLE 6
Baseline Clinical Characteristics.

| | Total | | Asymptomatic | | Symptomatic | | |
|---|---|---|---|---|---|---|---|
| | (n = 229) | | (n = 112) | | (n = 117) | | P Value |
| Age, years | 69.3 | (SD 8.3) | 67.2 | (SD 6.5) | 71.4 | (SD 9.2) | <0.005 |
| Body mass index | 26.9 | (SD 4.0) | 27.2 | (SD 3.9) | 26.6 | (SD 4.1) | NS |
| Gender men/women | 155/74 | | 75/37 | | 80/37 | | NS |
| Degree of stenosis, % | 90 | (IQR 80-95) | 88 | (IQR 81-95) | 83 | (IQR 75-95) | <0.05 |
| Diabetes mellitus, % | 38.9 | | 31.3 | | 46.2 | | <0.05 |
| Smoking, % | 33.6 | | 39 | | 28 | | NS |
| Hypertension, % | 75.5 | | 79.5 | | 71.8 | | NS |
| Fasting lipoproteins, mmol/L | | | | | | | |
| Total cholesterol | 4.3 | (IQR 3.5-5) | 4.4 | (SD 1.2) | 4.2 | (SD 1.0) | NS |
| LDL cholesterol | 2.5 | (IQR 1.9-3.1) | 2.4 | (IQR 1.8-3.0) | 2.6 | (IQR 2.0-3.3) | NS |
| HDL cholesterol | 1.2 | (IQR 0.9-1.3) | 1.2 | (IQR 0.9-1.4) | 1.2 | (IQR 0.9-1.3) | NS |
| Triglycerides | 1.5 | (IQR 1.0-1.8) | 1.5 | (IQR 0.9-1.9) | 1.4 | (IQR 1.0-1.7) | NS |
| Hemoglobin, g/L | 139.3 | (SD 13.7) | 141.3 | (SD 13.0) | 138.4 | (SD 14.2) | NS |
| CRP | 5.8 | (IQR 1.9-6.4) | 4.3 | (IQR 1.5-5.9) | 7.1 | (IQR 2.0-7.3) | NS |
| White blood cell count, 10⁹/L | 7.9 | (IQR 6.4-9) | 7.9 | (SD 2.0) | 7.8 | (SD 2.0) | NS |

| | | | | |
|---|---|---|---|---|
| Statins, % | 88.6 | 92.0 | 85.5 | NS |
| Anti-hypertensive treatment, (%) | 81.7 | 84.8 | 78.6 | NS |

Values are presented as mean and SD, or when not normally distributed as median with interquartile range (IQR). Significance between the symptomatic and asymptomatic group of patients are indicated with P value.

### Clinical Follow Up

The Swedish national inpatient health register was analyzed in order to identify postoperative cardiovascular (CV) events, with corresponding International Classification of Diseases, Tenth Revision (ICD-10) codes G45 and G46, 120 to 125 from October 2005 to December 2012. This is a nation-wide validated register where more than 99 percent of all somatic (including surgery) and psychiatric hospital discharges are registered. In doubtful cases, information was gained through telephone interviews and medical chart reviews. All causes of death were confirmed through the National Population Register.

### Definition of Outcomes

Each CV event was registered and analyzed separately. Events occurring in the first 24 hours postoperatively were considered as procedure-related and assumed as intraoperative for the analysis. Patients suffering more than one episode of the same event (for example, patients with multiple strokes) were classified as suffering multiple events. In these cases, only the first chronological event was taken into account in the survival analysis.

### Sample Preparation and Histology

Plaques were snap-frozen in liquid nitrogen immediately after surgical removal. Plaque homogenates were prepared as previously described. One mm fragments, from the most stenotic region, were taken for histology. Stainings for lipids (Oil Red O), vascular smooth muscle cells (VSMCs) (α-actin) and macrophages (CD68) were performed as previously described. Measurements of stained plaque area (%) for different stainings were quantified blindly using BiopixiQ 2.1.8 (Gothenburg, Sweden) after scanning with ScanScope Console Version 8.2 (LRI imaging AB, Vista Calif., USA).

### Assessment of Osteopontin, Thrombin Cleavage Osteopontin and Oxidized LDL in Plaque Homogenates

Plaque homogenate was centrifuged at 13,000×g for 10 minutes and 5 μL of the supernatant were used in each assay for measurement of osteopontin (IBL, cat. No. JP27158) and thrombin cleavage osteopontin (IBL, cat. No. JP27258) according to manufacture protocol.

### Cytokine Assessment

Aliquots of 50 μL of plaque homogenate were centrifuged at 13,000×g for 10 minutes. Twenty-five μL of the supernatant was removed and used for measuring fractalkine, interferon-γ (IFN-γ), interleukin-(IL)-6, monocyte chemoattractant protein-1 (MCP-1), monocyte inflammatory protein-1β (MIP-1β), PDGF-AB/BB, Regulated on Activation Normal T Cell Expressed and Secreted (RANTES), and tumor necrosis factor-α (TNF-α). The procedure was performed according to the manufacturer's instructions (Human Cytokine/chemokine immunoassay, Millipore Corporation, MA, USA) and analyzed with Luminex 100 IS 2.3 (Austin, Tex., USA).

### Statistics

All analyses were normalized against wet weight of the plaque. Values are presented as mean and standard deviation (SD) if normally distributed and as median and inter quartile range (IQR) if non-normally distributed. Two-group comparisons were performed with Mann-Whitney test if non-normally distributed variables, students t-test for normally distributed variables and χ2 test for categorical data. Spearman's rho was used for correlation analysis. Kaplan-Meier survival curves with Log rank test were used to analyze death, cardiovascular death and cardiovascular events free survival during follow-up. Differences were considered statistically significant at p<0.05.

### Results

Thrombin cleavage of OPN was associated with prevalent diabetes. Plaques from symptomatic patients with T2D diabetes showed higher levels of tcOPN compared with lesions from asymptomatic patients or patients without diabetes. Kaplan-Meier survival analysis showed that diabetic subjects in the highest tcOPN tertile experienced a significantly higher risk of cardiovascular death and total death. In the total cohort, the highest tcOPN tertile were associated with higher risk of death.

Accordingly, high levels of tcOPN in human carotid plaques were associated with symptoms prior to surgery and future cardiovascular death and total death in T2D diabetic subjects. In the total patient cohort, tcOPN levels were associated with death during follow-up.

The various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some preferred embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof

Preferred embodiments of this application are described herein, including the best mode known to the inventors for carrying out the application. Variations on those preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

We claim:

1. A composition comprising:
Osteopontin (OPN) or an antigenic fragment thereof; and
a protein carrier, wherein the protein carrier is bovine serum albumin (BSA), nonalbumin, edestin, exoprotein A from Pseudomonas aeruginosa, HC (hemocyanin from crab Paralithodes camtschatica), Helix promatia haemocyanin (HPH), human serum albumin (HSA), KTI (Kunits trypsin inhibitor from soybeans), keyhole limpet haemocyanin (KLH), LPH (haemocyanin from Limulus polyphemus), ovalbumin, Pam3Cys-Th, polylysine, porcine thyroglobulin (PTG), purified protein derivative (PPD), rabbit serum albumin (RSA), soybean trypsin inhibitor (STI) , sunflower globulin (SFG), tetanus toxoid, diphtheria toxoid, pan HLA DR-binding epitope (PADRE), Haemophilus influenza protein D, Neisseria meningitides outer membrane protein, flagellin, or CRM197.

2. The composition of claim 1, wherein the OPN or the antigenic fragment thereof is fused to the protein carrier.

3. The composition of claim 1, wherein the antigenic fragment of OPN comprises the sequence SLAYGLR (SEQ ID NO:1) and the protein carrier is PADRE (SEQ ID NO: 3).

4. The composition of claim 2, wherein the antigenic fragment consists of an amino acid sequence at least 85% identical to the amino acid sequence SLAYGLR (SEQ ID NO:1).

5. The composition of claim 2, wherein the antigenic fragment consists of an amino acid sequence at least 85% identical to the amino acid sequence GRGDSLAYGLR (SEQ ID NO:4).

6. The composition of claim 2, wherein the antigenic fragment consists of an amino acid sequence at least 85% identical to the amino acid sequence VDVPNGRGDSLAYGLR (SEQ ID NO:5).

7. The composition of claim 1, wherein the fragment of OPN comprises the sequence SVVYGLR (SEQ ID NO:2), and the protein carrier is PADRE comprising the sequence AKFVAAWTLKAAA (SEQ ID NO:3).

8. The composition of claim 2, wherein the antigen fragment of OPN consists of an amino acid sequence at least 85% identical to the amino acid sequence SVVYGLR (SEQ ID NO:2).

9. The composition of claim 2, wherein the antigenic fragment thereof consists of an amino acid sequence at least 85% identical to the amino acid sequence GRGDSVVYGLR (SEQ ID NO: 8).

10. The composition of claim 2, wherein the antigenic fragment thereof consists of an amino acid sequence at least 85% identical to the amino acid sequence VDTYDGRGDSVVYGLR (SEQ ID NO: 9).

11. The composition of claim 1, wherein the fragment of OPN induces an antibody response.

12. The composition of claim 1, further comprising an adjuvant and/or an excipient.

13. The composition of claim 12, wherein the adjuvant is an alum.

14. A method for treating a cardiovascular disease or an inflammation in a subject in need thereof, comprising: administering to the subject a therapeutically effect amount of the composition of claim 1.

15. The method of claim 14, wherein the administering is performed via an oral, nasal, subcutaneous, or intramuscular route of administration.

16. The method of claim 14 for treating atherosclerosis-related cardiovascular disease.

17. The method of claim 14 for treating an inflammation.

18. The method of claim 14, wherein the subject has, or shows symptoms of, type 2 diabetes.

19. A method of treating, reducing the likelihood or severity of, or slowing the progression of cardiovascular disease in a subject with type 2 diabetes, comprising:
detecting an elevated level of thrombin-cleaved osteopontin (tcOPN) in a carotid plaque of the subject; and
administering a therapeutically effective amount of the composition of claim 1 to the subject, thereby treating, reducing the likelihood or severity of, or slowing the progression of cardiovascular disease.

20. The method of claim 19, wherein the elevated level of tcOPN is higher than an averaged value obtained from subjects without diabetes or from asymptomatic subjects.


SEQUENCE LISTING (9 sequences)

SEQ ID NO: 1 - protein, 7 aa, Mouse
SLAYGLR

SEQ ID NO: 2 - protein, 7 aa, Homo sapiens
SVVYGLR

SEQ ID NO: 3 - protein, 13 aa, Artificial Sequence
  PADRE
AKFVAAWTLKAAA

SEQ ID NO: 4 - protein, 11 aa, Mouse
GRGDSLAYGLR

SEQ ID NO: 5 - protein, 16 aa, Mouse
VDVPNGRGDSLAYGLR

SEQ ID NO: 6 - protein, 24 aa, Artificial Sequence
  PADRE-tcOPN short
AKFVAAWTLKAAAGRGDSLAYGLR

SEQ ID NO: 7 - protein, 29 aa, Artificial Sequence
  PADRE-tcOPN long
AKFVAAWTLKAAAVDVPNGRGDSLAYGLR

SEQ ID NO: 8 - protein, 11 aa, Homo sapiens
GRGDSVVYGLR

SEQ ID NO: 9 - protein, 16 aa, Homo sapiens
VDTYDGRGDSVVYGLR